SEQUENCE LISTING (34 sequences)

SEQ ID NO: 1 - skipped

SEQ ID NO: 2 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 4 - protein, 5 aa, synthetic construct
GYTMN

SEQ ID NO: 5 - protein, 17 aa, synthetic construct
LINPYNSDTNYAQKLQG

SEQ ID NO: 6 - protein, 9 aa, synthetic construct
VALRVALDY

SEQ ID NO: 7 - protein, 11 aa, synthetic construct
KASQNVATHVG

SEQ ID NO: 8 - protein, 7 aa, synthetic construct
SASYRYS

SEQ ID NO: 9 - protein, 9 aa, synthetic construct
QQYNRYPYT

SEQ ID NO: 10 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 11 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 12 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 5 aa, synthetic construct
TYAMN

SEQ ID NO: 15 - protein, 19 aa, synthetic construct
RIRSKYNNYATYYAASVKG

SEQ ID NO: 16 - protein, 14 aa, synthetic construct
HGNFGNSYVSWFAY

SEQ ID NO: 17 - protein, 14 aa, synthetic construct
RSSTGAVTTSNYAN

SEQ ID NO: 18 - protein, 7 aa, synthetic construct
GTNKRAP

SEQ ID NO: 19 - protein, 9 aa, synthetic construct
ALWYSNLWV

SEQ ID NO: 20 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 21 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 22 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 23 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24-33 - skipped (10 entries)

SEQ ID NO: 34 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0356950 A1
(43) Pub. Date: Nov. 12, 2026

(54) METHODS FOR TREATING MULTIPLE MYELOMA

(71) Applicant: JANSSEN BIOTECH, INC., Horsham, PA (US)

(72) Inventors: Colleen KANE, Spring House, PA (US); Marie-Anne Damiette SMIT, Leiden (NL); Brandi HILDER, Spring House, PA (US); Jaszianne TOLBERT, Spring House, PA (US)

(21) Appl. No.: 19/487,330

(22) PCT Filed: May 31, 2024
(86) PCT No.: PCT/IB2024/055343

§ 371 Date: Nov. 24, 2025

Related U.S. Application Data
(60) Provisional application No. 63/505,640, filed on Jun. 1, 2023.
(60) Provisional application No. 63/588,520, filed on Oct. 6, 2023.
(60) Provisional application No. 63/594,771, filed on Oct. 31, 2023.

(51) Int. Cl. C07K 16/28; A61K 39/00; A61P 35/00; C07K 16/46

(52) U.S. Cl. CPC C07K 16/2809; A61P 35/00; C07K 16/28; C07K 16/468; A61K 2039/505; A61K 2039/545; C07K 2317/31; C07K 2317/52; C07K 2317/565; C07K 2317/71

(57) ABSTRACT

Embodiments of the present invention relate to methods of treating multiple myeloma in a subject in need thereof, comprising administering therapeutically effective amounts of a GPRC5DxCD3 bispecific antibody on a monthly dosing schedule.

### CROSS-REFERENCE TO RELATED APPLICATIONS

This application claims the benefit of priority of U.S. Provisional Application No. 63/505,640, filed Jun. 1, 2023, U.S. Provisional Application No. 63/588,520, filed Oct. 6, 2023, and U.S. Provisional Application No. 63/594,771, filed Oct. 31, 2023, the entire contents of which are incorporated herein by reference in their entirety.

### REFERENCE TO SEQUENCE LISTING SUBMITTED ELECTRONICALLY

This application contains a sequence listing, which is submitted electronically in XML format and is hereby incorporated by reference in its entirety. Said XML copy, created on May 8, 2024, is named “258199061602 (JBI6818WOPCT1) Sequence Listing.xml” and is 25,444 bytes in size.

### FIELD OF THE INVENTION

Methods of treating multiple myeloma are disclosed.

### BACKGROUND OF THE INVENTION

Multiple myeloma (MM) is a cancer of the plasma cells. Mechanistically, multiple myeloma is characterized by production of monoclonal proteins (M-proteins) comprised of pathological immunoglobulins or fragments of such, which have lost their function. The proliferation of multiple myeloma cells leads to subsequent displacement from the normal bone marrow niche, while overproduction of M-proteins causes characteristic osteolytic lesions, increased susceptibility to infections, hypercalcemia, renal insufficiency or failure, and neurological complications.

Treatment options for multiple myeloma have improved over time and vary depending on the aggressiveness of the disease, underlying prognostic factors, physical condition of the patient, and existing comorbidities. Therapeutic options include proteasome inhibitors (PIs), immunomodulatory drugs (IMiDs), alkylating agents, monoclonal antibodies (mAbs), antibody drug conjugate, histone deacetylase inhibitor, nuclear protein export inhibitor, chimeric antigen receptor (CAR) T cell therapy and stem cell transplantation.

Despite these therapeutic achievements, the disease recurs and is associated with additional risk factors (e.g., comorbidities or increasing age), thus warranting the need for novel therapeutic approaches, such as new dosage and treatment regimens. In particular in the elderly population, for which stem cell transplantation is often not a viable option, and in patients with refractory disease who exhausted several therapies, multiple myeloma remains an incurable malignancy and an unmet medical need with significant morbidity and mortality. In particular, there remains a need for therapeutic regimens that achieve rapid, deep and durable clinical responses, while providing manageable safety profiles.

### SUMMARY OF THE INVENTION

An embodiment of the present invention provides a method of treating multiple myeloma in a subject in need thereof, comprising administering to the subject a therapeutically effective amount of a GPRC5DxCD3 bispecific antibody on a monthly (Q4W) dosing schedule.

In certain embodiments, the GPRC5DxCD3 bispecific antibody comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the GPRC5D binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the GPRC5DxCD3 bispecific antibody is an IgG1, an IgG2, an IgG3 or an IgG4 isotype.

In certain embodiments, the GPRC5DxCD3 bispecific antibody is an IgG4 isotype.

In certain embodiments, the GPRC5DxCD3 bispecific antibody comprises one or more substitutions in its Fc region.

In certain embodiments, the GPRC5DxCD3 bispecific antibody is an IgG4 isotype and comprises Proline/Alanine/Alanine substitutions at amino acid positions 228/234/235, respectively, in its Fc region (according to EU index numbering).

In certain embodiments, the GPRC5DxCD3 bispecific antibody is an IgG4 isotype and comprises F405L and R409K substitutions in its Fc region (according to EU index numbering).

In certain embodiments, the Fc region of the GPRC5D binding arm comprises Proline/Alanine/Alanine substitutions at amino acid positions 228/234/235, respectively, in its Fc region (according to EU index numbering).

In certain embodiments, the Fc region of the CD3 binding arm comprises S228P, F234A, L235A, F405L, and R409K substitutions in its Fc region (according to EU index numbering).

In certain embodiments, the GPRC5DxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the GPRC5DxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the GPRC5DxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the GPRC5DxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the GPRC5DxCD3 bispecific antibody is talquetamab.

In certain embodiments, the subject has relapsed or refractory multiple myeloma.

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In certain embodiments, the method comprises subcutaneously administering treatment doses of the GPRC5DxCD3 bispecific antibody on the monthly dosing schedule (Q4W).

In certain embodiments, the GPRC5DxCD3 bispecific antibody (e.g., talquetamab) is administered as a monotherapy.

In certain embodiments, the GPRC5DxCD3 bispecific antibody (e.g., talquetamab) is administered as part of a combination therapy with one or more additional anti-myeloma agents.

In certain embodiments, the method comprises subcutaneously administering to the subject one or more step-up doses of the GPRC5DxCD3 bispecific antibody prior to administering the first treatment dose of the GPRC5DxCD3 bispecific antibody.

In certain embodiments, the method comprises subcutaneously administering the GPRC5DxCD3 bispecific antibody on the monthly dosing schedule (Q4W) at a treatment dose of from about 400 μg/kg to about 800 μg/kg.

In certain embodiments, the method comprises subcutaneously administering the GPRC5DxCD3 bispecific antibody on the monthly dosing schedule (Q4W) at a treatment dose of about 400 μg/kg.

In certain embodiments, the method comprises subcutaneously administering the GPRC5DxCD3 bispecific antibody on the monthly dosing schedule (Q4W) at a treatment dose of about 800 μg/kg.

In certain embodiments, the method comprises subcutaneously administering to the subject at least one treatment dose of the GPRC5DxCD3 bispecific antibody on a bi-weekly dosing schedule (Q2W) and subsequently administering to the subject treatment doses of the GPRC5DxCD3 bispecific antibody on the monthly dosing schedule (Q4W).

In certain embodiments, the method comprises subcutaneously administering to the subject at least one treatment dose of the GPRC5DxCD3 bispecific antibody on a weekly dosing schedule (QW) and then subcutaneously administering to the subject at least one treatment dose of the GPRC5DxCD3 bispecific antibody on a bi-weekly dosing schedule (Q2W) and then subsequently administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 6 treatment cycles, and starting at treatment cycle 7, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 6 treatment cycles, and if the patient has achieved a VGPR, CR or sCR, as determined by IMWG response criteria, then starting at treatment cycle 7, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 7 treatment cycles, and starting at treatment cycle 8, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 7 treatment cycles, and if the patient has achieved a VGPR, CR or sCR, as determined by IMWG response criteria, then starting at treatment cycle 8, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 8 treatment cycles, and starting at treatment cycle 9, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 8 treatment cycles, and if the patient has achieved a VGPR, CR or sCR, as determined by IMWG response criteria, then starting at treatment cycle 9, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 7 or treatment cycle 8.

In certain embodiments, the method comprises subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 5 if the subject has achieved a complete response or a stringent complete response, as determined by IMWG response criteria.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of a clinical response in the subject.

In certain embodiments, each treatment dose amount of the GPRC5DxCD3 bispecific antibody administered on a weekly dosing schedule (QW), a bi-weekly dosing schedule (Q2W) or the monthly dosing schedule (Q4W), is the same.

In certain embodiments, the GPRC5DxCD3 bispecific antibody is administered on the bi-weekly dosing schedule (Q2W) and the monthly dosing schedule (Q4W), at a treatment dose amount of 800 μg/kg.

In certain embodiments, the method comprises subcutaneously administering 2 or 3 step-up doses of the GPRC5DxCD3 bispecific antibody prior to subcutaneously administering the first treatment dose.

In certain embodiments, the method comprises subcutaneously administering step-up doses of 10 μg/kg and 60 μg/kg of the GPRC5DxCD3 bispecific antibody prior to subcutaneously administering a treatment dose.

In certain embodiments, the method comprises subcutaneously administering step-up doses of 10 μg/kg and 60 μg/kg and 400 μg/kg of the GPRC5DxCD3 bispecific antibody prior to subcutaneously administering a treatment dose.

In certain embodiments, the method comprises subcutaneously administering step-up doses of the GPRC5DxCD3 bispecific antibody 2-4 days apart from each other.

In certain embodiments, the subject achieves a clinical response that is a PR, or a VGPR, or a CR or a sCR.

In certain embodiments, the subject achieves a clinical response that is a CR or a sCR.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

In certain embodiments, the method comprises subcutaneously administering 2 or 3 step-up doses of the GPRC5DxCD3 bispecific antibody prior to subcutaneously administering the first treatment dose.

In certain embodiments, the method comprises subcutaneously administering step-up doses of 10 μg/kg and 60 μg/kg of the GPRC5DxCD3 bispecific antibody prior to subcutaneously administering a treatment dose.

In certain embodiments, the method comprises subcutaneously administering step-up doses of 10 μg/kg and 60 μg/kg and 400 μg/kg of the GPRC5DxCD3 bispecific antibody prior to subcutaneously administering a treatment dose.

In certain embodiments, the method comprises subcutaneously administering step-up doses of the GPRC5DxCD3 bispecific antibody 2-4 days apart from each other.

In certain embodiments, the method comprises treating the subject according to a therapeutically effective regimen that comprises sequential 28-day GPRC5DxCD3 treatment cycles, wherein: one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W).

In certain embodiments, the method comprises treating the subject according to a therapeutically effective regimen that comprises sequential 28-day GPRC5DxCD3 treatment cycles, wherein: one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

In certain embodiments, each treatment dose of the GPRC5DxCD3 bispecific antibody administered on the bi-weekly dosing schedule (Q2W) and the monthly dosing schedule (Q4W) is 800 μg/kg.

In certain embodiments, Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W).

In certain embodiments, Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

In certain embodiments, 1-3 step-up doses of the GPRC5DxCD3 bispecific antibody are administered to the subject during the step-up phase.

In certain embodiments, two step-up doses of the GPRC5DxCD3 bispecific antibody are administered to the subject during the step-up phase.

In certain embodiments, three step-up doses of the GPRC5DxCD3 bispecific antibody are administered to the subject during the step-up phase.

In certain embodiments, the step-up phase comprises a first step-up dose of 0.01 mg/kg, a second step-up dose of 0.06 mg/kg, and a third step-up dose of 0.4 mg/kg.

In certain embodiments, one or more treatment doses of the GPRC5DxCD3 bispecific antibody are subcutaneously administered to the subject during the step-up phase, in addition to the one or more step-up doses (e.g., on a weekly dosing schedule).

In certain embodiments, one or two treatment doses of the GPRC5DxCD3 bispecific antibody are subcutaneously administered to the subject during the step-up phase, in addition to the one or more step-up doses (e.g., on a weekly dosing schedule).

In certain embodiments, Cycle 1 of the regimen comprises a first step-up dose of 0.01 mg/kg, a second step-up dose of 0.06 mg/kg, a third step-up dose of 0.4 mg/kg, and a treatment dose of 0.8 mg/kg.

In certain embodiments, Cycle 1 of the regimen comprises a first step-up dose of 0.01 mg/kg, a second step-up dose of 0.06 mg/kg 2-4 days after the first step-up dose, a third step-up dose of 0.4 mg/kg 4-7 days after the second step-up dose, and a treatment dose of 0.8 mg/kg 5-9 days after the third step-up dose.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15.

In certain embodiments, each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1.

In certain embodiments, either: (i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and each of Cycles 2-6 of the regimen comprises a Q2W treatment dose of 0.8 mg/kg on Day 1 and Day 15; and each of Cycles 7+ comprises a Q4W treatment dose of 0.8 mg/kg on Day 1.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and either: (i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

In certain embodiments, a dosing regimen comprising administration of the GPRC5DxCD3 bispecific antibody on the monthly (Q4W) dosing schedule improves (i) the clinical response rate (e.g., ORR) and/or (ii) the rate and/or severity of adverse events in a population of subjects compared to a reference population of subjects that receives the GPRC5DxCD3 bispecific antibody on a weekly (QW) and/or bi-weekly (Q2W) dosing schedule and not a monthly (Q4W) dosing schedule.

In certain embodiments, a dosing regimen comprising the monthly (Q4W) dosing schedule achieves a lower rate and/or lesser duration of adverse events than a dosing regimen comprising a weekly (QW) and/or bi-weekly (Q2W) dosing schedule and not a monthly (Q4W) dosing schedule.

In certain embodiments, the adverse events comprise one or more of oral toxicity (e.g., ageusia, dysgeusia, dry mouth, and/or dysphagia), and/or nail toxicity (e.g., nail disorder), and/or skin toxicity (e.g., dry skin, skin exfoliation, and/or pruritus).

In certain embodiments, a dosing regimen comprising the monthly (Q4W) dosing schedule achieves a stronger and/or longer duration of clinical response than a dosing regimen comprising a weekly (QW) and/or bi-weekly (Q2W) dosing schedule and not a monthly (Q4W) dosing schedule. In certain embodiments, a dosing regimen comprising the monthly (Q4W) dosing schedule achieves a higher overall response rate (ORR) than a dosing regimen comprising a weekly (QW) and/or bi-weekly (Q2W) dosing schedule and not a monthly (Q4W) dosing schedule. In certain embodiments, a dosing regimen comprising the monthly (Q4W) dosing schedule achieves longer progression-free survival (PFS) than a dosing regimen comprising a weekly (QW) and/or bi-weekly (Q2W) dosing schedule and not a monthly (Q4W) dosing schedule.

All methods described herein, however expressed, may be described as corresponding uses, in particular medical uses.

### DETAILED DESCRIPTION OF THE INVENTION

The disclosed methods can be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 5%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

“Antigen binding fragment” or “antigen binding domain” refers to a portion of an immunoglobulin molecule that binds an antigen. Antigen binding fragments can be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 2. SEQ ID NO: 3 shows the extracellular domain of CD3 epsilon.

SEQ ID NO: 2
MQSGTHWRVLGLCLLSVGVWGQDGNEEMGGITQTPYKVSISGTTVILTCPQ
YPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSK
NRKAKAKPVTRGAGAGGRQRGQNKERPPPVPNPDYEPIRKGQRDLYSGLNQ
RRI
SEQ ID NO: 3
DGNEEMGGITQTPYKVSISGTTVILTCPQYPGSEILWQHNDKNIGGDEDDK
NIGSDEDHLSLKEFSELEQSGYYVCYPRGSKPEDANFYLYLRARVCENCME
MD

“CH3 region” or “CH3 domain” refers to the CH3 region of an immunoglobulin. The CH3 region of human IgG1 antibody corresponds to amino acid residues 341-446. However, the CH3 region can also be any of the other antibody isotypes as described herein.

“Combination” means that two or more therapeutics are administered to a subject together in a mixture, concurrently as single agents or sequentially as single agents in any order.

“Combination dosing regimen” (also referred to as “combination regimen” or “combination therapy”) as used herein refers to a therapeutically effective regimen that comprises administration of two or more anti-multiple myeloma therapeutic agents to a subject to treat multiple myeloma. The two or more therapeutics are administered to a subject on each therapeutic's respective dosing schedule over a time period (e.g., a time period may comprise one or more treatment cycles, such as one or more 28-day treatment cycles); for example, a combination dosing regimen may comprise administering to a subject(i) “therapeutic #1” on its weekly or bi-weekly or monthly dosing schedule starting on Day 1 of a treatment cycle and (ii) “therapeutic #2” on its weekly or bi-weekly or monthly dosing schedule starting on Day 1 of the same treatment cycle or a subsequent treatment cycle.

“Complementarity determining regions” (CDR) are antibody regions that bind an antigen. CDRs can be defined using various delineations such as Kabat(Wu et al. J Exp Med 132:211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196:901-17, 1987), IMGT (Lefranc et al. Dev Comp Immunol 27:55-77, 2003) and AbM (Martin and Thornton J Bmol Biol 263:800-15, 1996). The correspondence between the various delineations and variable region numbering are described (see e.g. Lefranc et al. Dev Comp Immunol 27:55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC can be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Preferably, the term “CDR”, “HCDR1”, “HCDR2”, “HCDR3”, “LCDR1”, “LCDR2” and “LCDR3” as used herein includes CDRs defined by the method of Kabat.

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Fc gamma receptor” (FcγR) refers to well-known FcγRI, FcγRIIa, FcγRIIb or FcγRIII. Activating FcγR includes FcγRI, FcγRIIa and FcγRIII.

“GPRC5DxCD3 bispecific antibody” refers to a bispecific antibody that specifically binds GPRC5D and CD3.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks can not be exact copies of expressed human immunoglobulin or human immunoglobulin germline gene sequences.

“Identity” refers to a relationship between the sequences of two or more polypeptide molecules or two or more nucleic acid molecules, as determined by aligning and comparing the sequences. “Percent(%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies can have heterogeneous glycosylation within the antibody population. Monoclonal antibody can be monospecific or multispecific such as bispecific, monovalent, bivalent or multivalent.

“Mutation” refers to an engineered or naturally occurring alteration in a polypeptide or polynucleotide sequence when compared to a reference sequence. The alteration can be a substitution, insertion or deletion of one or more amino acids or polynucleotides.

“Negative minimal residual disease status” or “negative MRD status” or “MRD negative” refers to the PerMillionCount(i.e., a point estimate of malignant myeloma cells per million nucleated cells) in a patients on-study bone marrow sample relative to their reference bone marrow sample (i.e., talquetamab treatment naïve bone marrow sample). Based on this PerMillionCount, each sample is determined to be positive or negative. Samples are positive if the PerMillionCount is greater than or equal to the limit of sensitivity, otherwise they are negative. Negative minimal residual disease status can be determined at a sensitivity of 0.01% (10-4), 0.001% (10-5) or 0.0001% (10-6). Negative minimal residual disease status was determined using next generation sequencing (NGS).

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Recombinant” refers to DNA, antibodies and other proteins that are prepared, expressed, created or isolated by recombinant means when segments from different sources are joined to produce recombinant DNA, antibodies or proteins.

“Refractory” refers to a cancer that is not amendable to surgical intervention and is initially unresponsive to therapy.

“Relapsed” refers to a cancer that responded to treatment but then returns.

“Step-up dose” refers to a dose of an active agent that is administered to a subject prior to a treatment dose. A step-up dose is lower than the treatment dose. To prevent or lessen certain toxicities, such as cytokine release syndrome (CRS), a “priming” dose strategy may include one or more lower step-up dose(s) followed by higher treatment doses. A “step-up phase” refers to an initial phase of a therapeutically effective regimen in which at least one step-up dose of a therapeutic is administered to the subject. A step-up phase may also include one or more treatment doses, i.e., a step-up phase may include one or more step-up doses followed by one or more treatment doses; for example, a step-up phase may include two step-up doses followed by two treatment doses. In particular embodiments, the step-up phase is 28 days, i.e., the step-up phase is a 28-day cycle of a therapeutically effective regimen.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as GPRC5D. Examples of T cell antigen include, e.g., CD3. This dual/multi-target binding ability recruits T cells to the target cell or tissue leading to the eradication of the target cell or tissue.

“Therapeutically effective amount” refers to an amount effective, at doses and for periods of time necessary, to achieve a desired therapeutic result. A therapeutically effective amount can vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Treat” or “treatment” refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Treatment dose”-refers to a dose of the active agent that is administered to a subject to treat a disease. A treatment dose may be administered at a regular dosing interval on a repetitive basis (e.g. weekly, biweekly, monthly). A treatment dose may be preceded by one or more step-up doses.

A patient that is “triple-class exposed” refers to a patient diagnosed with multiple myeloma (MM) that has previously been treated with (at a minimum) a proteasome inhibitor, an immunomodulatory agent and an anti-CD38 monoclonal antibody.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### GPRC5DxCD3 Bispecific Antibodies and Uses Thereof

It is well-known in the art that drug development is an unpredictable field. The lack of predictability in the art is evidenced, for example, by health authority requirements (such as those of the Food and Drug Administration) to establish a safe and effective dosing regimen for each individual drug candidate in clinical trials. Over the last decade (2011-2020), only 7.9% of all developmental drug candidates achieved FDA approval from a Phase I clinical study. See Clinical Development Success Rates and Contributing Factors 2011-2020. The rate of success is even lower in oncology, such that only 5.3% of oncology drug candidates succeed.

In the field of oncology, even for a drug that already has an established dose in a particular indication, the Food and Drug Administration (FDA) recommends further clinical studies to identify an optimal dose for a new indication; otherwise, patients may be exposed to unreasonable and significant risk, among other potential deficiencies. See, e.g., Optimizing the Dosage of Human Prescription Drugs and Biological Products for the Treatment of Oncologic Diseases; Draft Guidance for Industry; January 2023).

In patients with relapsed or refractory disease who have exhausted several therapies, multiple myeloma remains an incurable malignancy and an unmet medical need with significant morbidity and mortality. The present inventors have developed novel dosing regimens for GPRC5DxCD3 bispecific antibodies that provide improved safety profiles over currently approved regimens while maintaining deep and durable efficacy.

The inventors have discovered that adverse events (AEs) associated with the GPRC5D target, such as oral toxicity (e.g., ageusia, dysgeusia, dry mouth, and/or dysphagia), and nail toxicity (e.g., nail disorder), and skin toxicity (e.g., dry skin, skin exfoliation, and/or pruritus) can be mitigated by certain dosing schedules that comprise less frequent dosing of talquetamab, while still achieving efficacious responses. It was also discovered that sufficient exposure to talquetamab, and minimal time to best response, can be achieved after about four 28-day treatment cycles, in accordance with dosing schedules described herein, so that patients can experience reduced AEs while still having deep and durable responses and minimal amount of time to clinical response.

Monthly (Q4W) dosing of talquetamab was selected based on the pharmacokinetic (PK), pharmacodynamic, safety, and efficacy findings from a subset of the pivotal RP2D population in the MonumtanTAL-1 clinical study who switched to less frequent dosing (e.g., as described in the Example section herein), the Phase 1b TriMM-2 clinical study (combination therapy in relapsed/refractory populations), and early data from MonumenTAL-2 (combination therapy in relapsed/refractory populations). In those studies, TEAEs were tolerable and manageable; CRS was generally low grade and neurotoxicity events were rare. Additionally, data from MonumenTAL-1 (e.g., as described in the Example section herein) demonstrates that participants who switched to less frequent dosing after achieving response with talquetamab monotherapy have improved progression-free survival (PFS) compared with the overall pivotal population and experienced fewer study drug-related TEAEs and Grade 3 or 4 TEAEs after switching and experienced improve resolution of on-target-related toxicity (e.g., oral, skin, and rash AEs). Overall, data from MonumanTAL-1 in which participants are receiving Q4W dosing demonstrated maintenance of response following switch, with a trend for improved resolution of GPRC5D-related oral, skin (rash and non-rash), and nail toxicities.

PK simulations showed that talquetamab trough levels following the first 0.8 mg/kg SC administration were comparable or higher than the maximum EC₉₀ values identified in an ex vivo cytotoxicity assay. This assay assessed the ability of talquetamab to induce killing using mononuclear cells from the bone marrow samples of multiple myeloma patients in co-culture with T cells from healthy donors. The trough concentrations at steady state were also comparable to or higher than the maximum EC₉₀. It is believed that embodiments of the Q4W dosing schedule disclosed herein would maintain talquetamab's efficacy by balancing maximal reduction of disease burden with biweekly dosing in the initial 6 cycles of treatment with participant convenience and reduced exposure with Q4W dosing from Cycle 7 onward for participants with response of confirmed PR or better (or as early as Cycle 5 for participants who have achieved a confirmed response of VGPR or better).

### Antibodies of the Present Invention

Any suitable GPRC5DxCD3 bispecific antibody known to those skilled in the art in view of the present disclosure can be used in the invention.

Various bispecific antibody formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv-and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or bispecific antibodies generated by arm exchange. Exemplary bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), DuoBody (Genmab), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12:276 and in Nunez-Prado et al., (2015) Drug Discovery Today 20 (5): 588-594.

In some embodiments, the GPRC5DxCD3 bispecific antibody comprises any one of the GPRC5D binding domains described in U.S. Pat. No. 10,562,968, the content of which is incorporated herein by reference in its entirety. In some embodiments, the GPRC5DxCD3 bispecific antibody comprises any one of the CD3 binding domains described in U.S. Pat. No. 10,562,968. In some embodiments, the GPRC5DxCD3 bispecific antibody comprises any one of the GPRC5DxCD3 bispecific antibodies described in U.S. Pat. No. 10,562,968.

In some embodiments, the GPRC5DxCD3 bispecific antibody is chimeric, humanized or human.

In some embodiments, the bispecific antibody is an IgG1, an IgG2, an IgG3 or an IgG4 isotype. In preferred embodiments, the bispecific antibody is an IgG4 isotype. An exemplary wild-type IgG4 comprises an amino acid sequence of SEQ ID NO: 34.

SEQ ID NO: 34:
ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK

The bispecific antibody can be of any allotype. It is expected that allotype has no influence on properties of the bispecific antibodies, such as binding or Fc-mediated effector functions. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host can be determined in part by the allotype of the antibody (Stickler et al., (2011) Genes and Immunity 12:213-21). Antibody allotype is related to amino acid sequence variations at specific locations in the constant region sequences of the antibody. Table 2 shows select IgG1, IgG2 and IgG4 allotypes.

TABLE 2

| | Amino acid residue at position of | | | | | | | |
|---|---|---|---|---|---|---|---|---|
| | diversity (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n− | T | V | | | | | | |
| nG4m(a) | | | L | R | | | | |
| G1m(17) | | | | | K | E | M | A |
| G1m(17,1) | | | | | K | D | L | A |

In some embodiments, the bispecific antibody comprises one or more Fc substitutions that reduces binding of the bispecific antibody to a Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP). The specific substitutions can be made in comparison to the wild-type IgG4 of SEQ ID NO: 34.

Fc positions that can be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4, wherein residue numbering is according to the EU index.

Fc substitutions that can be used to reduce CDC are a K322A substitution.

Well-known S228P substitution can further be made in IgG4 antibodies to enhance IgG4 stability.

In some embodiments, the bispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F405L/K409R, wild-type/F405L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the GPRC5DxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the GPRC5DxCD3 bispecific antibody further comprises proline at position 228, alanine at position 234 and alanine at position 235 in both the HC1 and the HC2.

Tables 3 and 4 provide sequences of an exemplary embodiment of a GPRC5DxCD3 bispecific antibody, according to the Kabat numbering system.

TABLE 3
Sequences of GPRC5D binding arm

| | | SEQ ID |
|---|---|---|
| Region | Sequence | NO: |
| HCDR1 | GYTMN | 4 |
| HCDR2 | LINPYNSDTNYAQKLQG | 5 |
| HCDR3 | VALRVALDY | 6 |
| LCDR1 | KASQNVATHVG | 7 |
| LCDR2 | SASYRYS | 8 |
| LCDR3 | QQYNRYPYT | 9 |
| VH | QVQLVQSGAEVKKPGASVKVSCKASGYSF | 10 |
| | TGYTMNWVRQAPGQGLEWMGLINPYNSD | |
| | TNYAQKLQGRVTMTTDTSTSTAYMELRSL | |
| | RSDDTAVYYCARVALRVALDYWGQGTLV | |
| | TVSS | |
| VL | DIQMTQSPSSLSASVGDRVTITCKASQNVA | 11 |
| | THVGWYQQKPGKAPKRLIYSASYRYSGVP | |
| | SRFSGSGSGTEFTLTISNLQPEDFATYYCQQ | |
| | YNRYPYTFGQGTKLEIK | |
| HC | QVQLVQSGAEVKKPGASVKVSCKASGYSF | 12 |
| | TGYTMNWVRQAPGQGLEWMGLINPYNSD | |
| | TNYAQKLQGRVTMTTDTSTSTAYMELRSL | |
| | RSDDTAVYYCARVALRVALDYWGQGTLV | |
| | TVSSASTKGPSVFPLAPCSRSTSESTAALGC | |
| | LVKDYFPEPVTVSWNSGALTSGVHTFPAV | |
| | LQSSGLYSLSSVVTVPSSSLGTKTYTCNVD | |
| | HKPSNTKVDKRVESKYGPPCPPCPAPEAAG | |
| | GPSVFLFPPKPKDTLMISRTPEVTCVVVDV | |
| | SQEDPEVQFNWYVDGVEVHNAKTKPREE | |
| | QFNSTYRVVSVLTVLHQDWLNGKEYKCK | |
| | VSNKGLPSSIEKTISKAKGQPREPQVYTLPP | |
| | SQEEMTKNQVSLTCLVKGFYPSDIAVEWE | |
| | SNGQPENNYKTTPPVLDSDGSFFLYSRLTV | |
| | DKSRWQEGNVFSCSVMHEALHNHYTQKS | |
| | LSLSLGK | |
| LC | DIQMTQSPSSLSASVGDRVTITCKASQNVA | 13 |
| | THVGWYQQKPGKAPKRLIYSASYRYSGVP | |
| | SRFSGSGSGTEFTLTISNLQPEDFATYYCQQ | |
| | YNRYPYTFGQGTKLEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |

TABLE 4
Sequences of CD3 binding arm

| | | SEQ |
|---|---|---|
| Region | Sequence | ID NO: |
| HCDR1 | TYAMN | 14 |
| HCDR2 | RIRSKYNNYATYYAASVKG | 15 |
| HCDR3 | HGNFGNSYVSWFAY | 16 |
| LCDR1 | RSSTGAVTTSNYAN | 17 |
| LCDR2 | GTNKRAP | 18 |
| LCDR3 | ALWYSNLWV | 19 |
| VH | EVQLVESGGGLVQPGGSLRLSCAASGFTFNT | 20 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSS | |
| VL | QTVVTQEPSLTVSPGGTVTLTCRSSTGAVTT | 21 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQP | |
| HC | EVQLVESGGGLVQPGGSLRLSCAASGFTFNT | 22 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSSASTKGPSVFPLAPCSRSTSESTAALGC | |
| | LVKDYFPEPVTVSWNSGALTSGVHTFPAVL | |
| | QSSGLYSLSSVVTVPSSSLGTKTYTCNVDHK | |
| | PSNTKVDKRVESKYGPPCPPCPAPEAAGGPS | |
| | VFLFPPKPKDTLMISRTPEVTCVVVDVSQED | |
| | PEVQFNWYVDGVEVHNAKTKPREEQFNSTY | |
| | RVVSVLTVLHQDWLNGKEYKCKVSNKGLPS | |
| | SIEKTISKAKGQPREPQVYTLPPSQEEMTKNQ | |
| | VSLTCLVKGFYPSDIAVEWESNGQPENNYKT | |
| | TPPVLDSDGSFLLYSKLTVDKSRWQEGNVFS | |
| | CSVMHEALHNHYTQKSLSLSLGK | |
| LC | QTVVTQEPSLTVSPGGTVTLTCRSSTGAVTT | 23 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQPKAAPSVTLFP | |
| | PSSEELQANKATLVCLISDFYPGAVTVAWKA | |
| | DSSPVKAGVETTTPSKQSNNKYAASSYLSLT | |
| | PEQWKSHRSYSCQVTHEGSTVEKTVAPTECS | |

In some embodiments, the GPRC5DxCD3 bispecific antibody is JNJ-64407564 or Talquetamab (also referred to herein as Tal), which has the sequences described in Tables 3 and 4.

Talquetamab is a GPRC5D-directed bispecific antibody in development for treatment of patients with relapsed or refractory multiple myeloma. See, e.g., Chari A, et al. Blood 2022; 140 (suppl 1): 384-387, which is incorporated by reference herein. Talquetamab is a bispecific GPRC5D-directed CD3 T cell engager in development as a monotherapy for the treatment of adult patients with relapsed or refractory multiple myeloma who have received at least three or four prior therapies, including a proteasome inhibitor, an immunomodulatory agent and an anti-CD38 monoclonal antibody.

Talquetamab and its methods of use are described, for example, in WO 2018/017786 and WO 2022/058445, which are incorporated by reference herein. According to particular embodiments, the GPRC5DxCD3 bispecific antibody has an amino acid sequence with at least 80%, at least 85%, at least 90%, at least 95%, at least 98%, or at least 99% sequence identity to the amino acid sequence of talquetamab.

Additional embodiments of GPRC5DxCD3 bispecific antibodies that may be used in accordance with the present invention are described below.

In certain embodiments, the GPRC5DxCD3 bispecific antibody comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the GPRC5D binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the GPRC5DxCD3 bispecific antibody is an IgG1, an IgG2, an IgG3 or an IgG4 isotype.

In certain embodiments, the GPRC5DxCD3 bispecific antibody is an IgG4 isotype. In certain embodiments, the GPRC5DxCD3 bispecific antibody comprises one or more substitutions in its Fc region.

In certain embodiments, the GPRC5DxCD3 bispecific antibody is an IgG4 isotype and comprises S228P, F234A and L235A substitutions in its Fc region.

In certain embodiments, the GPRC5DxCD3 bispecific antibody is an IgG4 isotype and comprises S228P, F234A, L235A F405L and R409K substitutions in its Fc region.

In certain embodiments, the GPRC5D binding arm comprises S228P, F234A and L235A substitutions in its Fc region (according to EU index numbering).

In certain embodiments, the Fc region of the CD3 binding arm comprises S228P, F234A, L235A, F405L, and R409K substitutions in its Fc region (according to EU index numbering).

In certain embodiments, the GPRC5DxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the GPRC5DxCD3 bispecific antibody comprises a first heavy chain (HC1) having at least 90% identity to the amino acid sequence of SEQ ID NO:

12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the GPRC5DxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the GPRC5DxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the GPRC5DxCD3 bispecific antibody is talquetamab.

Patient Populations with Multiple Myeloma

The GPRC5DxCD3 bispecific antibodies disclosed herein are for use in treating multiple myeloma in a subject, for example a human subject. In certain embodiments, the subject is relapsed or refractory to treatment with one or more prior anti-cancer treatments. Relapsed disease means a cancer has come back. Refractory disease means a cancer has not improved with treatment or has stopped responding to treatment.

In some embodiments, the subject is relapsed or refractory to treatment with a therapeutic used to treat multiple myeloma or other hematological malignancies.

In certain embodiments, the subject has had been treated with from 1 to 11 prior lines of therapy, or from 1 to 10 prior lines of therapy.

In certain embodiments, the subject has previously received an autologous stem cell transplant(ASCT).

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In particular embodiments, the patients are relapsed or refractory or intolerant to the last line of therapy (LOT); were exposed to a proteasome inhibitor, immunomodulatory drug, and anti-CD38 therapy; and had measurable disease.

In some embodiments, the subject has received three anti-cancer therapies prior to administration of the GPRC5DxCD3 bispecific antibody.

In one embodiment, the three prior anti-cancer therapies are a proteasome inhibitor (PI), an immunomodulatory drug (ImiD), and an anti-CD38 antibody. In certain such embodiments, the proteasome inhibitor is bortezomib, carfilzomib or ixazomib, the immunomodulatory drug (ImiD) is lenalidomide, pomalidomide or thalidomide and the anti-CD38 antibody is daratumumab or isatuximab.

In one embodiment, the proteasome inhibitor is bortezomib, the immunomodulatory drug (ImiD) is lenalidomide and the anti-CD38 antibody is daratumumab. In one embodiment, the proteasome inhibitor is bortezomib, the immunomodulatory drug (ImiD) is lenalidomide and the anti-CD38 antibody is isatuximab. In one embodiment, the proteasome inhibitor is bortezomib, the immunomodulatory drug (ImiD) is pomalidomide and the anti-CD38 antibody is daratumumab. In one embodiment, the proteasome inhibitor is bortezomib, the immunomodulatory drug (ImiD) is pomalidomide and the anti-CD38 antibody is isatuximab. In one embodiment, the proteasome inhibitor is bortezomib, the immunomodulatory drug (ImiD) is thalidomide and the anti-CD38 antibody is daratumumab. In one embodiment, the proteasome inhibitor is bortezomib, the immunomodulatory drug (ImiD) is thalidomide and the anti-CD38 antibody is isatuximab.

In one embodiment, the proteasome inhibitor is carfilzomib, the immunomodulatory drug (ImiD) is lenalidomide and the anti-CD38 antibody is daratumumab. In one embodiment, the proteasome inhibitor is carfilzomib, the immunomodulatory drug (ImiD) is lenalidomide and the anti-CD38 antibody is isatuximab. In one embodiment, the proteasome inhibitor is carfilzomib, the immunomodulatory drug (ImiD) is pomalidomide and the anti-CD38 antibody is daratumumab. In one embodiment, the proteasome inhibitor is carfilzomib, the immunomodulatory drug (ImiD) is pomalidomide and the anti-CD38 antibody is isatuximab. In one embodiment, the proteasome inhibitor is carfilzomib, the immunomodulatory drug (ImiD) is thalidomide and the anti-CD38 antibody is daratumumab. In one embodiment, the proteasome inhibitor is carfilzomib, the immunomodulatory drug (ImiD) is thalidomide and the anti-CD38 antibody is isatuximab.

In one embodiment, the proteasome inhibitor is ixazomib, the immunomodulatory drug (ImiD) is lenalidomide and the anti-CD38 antibody is daratumumab. In one embodiment, the proteasome inhibitor is ixazomib, the immunomodulatory drug (ImiD) is lenalidomide and the anti-CD38 antibody is isatuximab. In one embodiment, the proteasome inhibitor is ixazomib, the immunomodulatory drug (ImiD) is pomalidomide and the anti-CD38 antibody is daratumumab. In one embodiment, the proteasome inhibitor is ixazomib, the immunomodulatory drug (ImiD) is pomalidomide and the anti-CD38 antibody is isatuximab. In one embodiment, the proteasome inhibitor is ixazomib, the immunomodulatory drug (ImiD) is thalidomide and the anti-CD38 antibody is daratumumab. In one embodiment, the proteasome inhibitor is ixazomib, the immunomodulatory drug (ImiD) is thalidomide and the anti-CD38 antibody is isatuximab.

In some embodiments, the subject is refractory or relapsed to treatment with one or more treatments or therapies, such as THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, or other BCMA-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In one embodiment, the anti-CD38 antibody is daratumumab.

In another embodiment, the anti-CD38 antibody is isatuximab

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4;14) (p16;q32), t(14;16) (q32;q23), del17p, 1qAmp, t(4;14) (p16;q32) and t(14;16) (q32;q23), t(4;14) (p16;q32) and del17p, t(14;16) (q32;q23) and del17p, or t(4;14) (p16;q32), t(14;16) (q32;q23) and del17p. In some embodiments, the subject having the high-risk multiple myeloma has one or more chromosomal abnormalities comprising: t(4;14) (p16;q32), t(14;16) (q32;q23), del17p, 1qAmp, t(4;14) (p16;q32) and t(14;16) (q32;q23), t(4;14) (p16;q32) and del17p, t(14;16) (q32;q23) and del17p; or t(4;14) (p16;q32), t(14;16) (q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. T (4;14) (p16;q32) involves translocation of fibroblast growth factor receptor 3 (FGFR3) and multiple myeloma SET domain containing protein (MMSET) (also called WHSC1/NSD2), and t(14;16) (q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

### Monthly Administration of a GPRC5DxCD3 Bispecific Antibody

The inventors have developed novel dosing regimens for GPRC5DxCD3 bispecific antibodies that provide improved safety profiles over currently approved regimens while maintaining deep and durable clinical responses over time. The regimens provide reduced or less frequent dosing, which mitigate the rate and/or severity of adverse events while maintaining clinical responses.

As used herein, “weight-based” refers to administration of a dose amount that is based on the subject's specific body weight; for example, 3 mg/kg refers to a dose of 3 milligrams of antibody per kilogram of the subject's body weight. Unless otherwise specified herein, when a dose is described in a unit of “mg/kg” or “μg/kg,” weight-based dosing is being employed.

Unless otherwise specified herein, a GPRC5DxCD3 bispecific antibody, such as talquetamab, is administered on a dosing schedule based on sequential 28-day cycles, for example, Cycle 1 starts on Day 1 of Cycle 1 and ends on Day 28 of Cycle 1, and then Day 1 of Cycle 2 starts the day after Day 28 of Cycle 1 and ends on Day 28 of Cycle 2, and then Day 1 of Cycle 3 starts the day after Day 28 of Cycle 2 and ends on Day 28 of Cycle 3, and so on. In certain embodiments, one or more step-up doses are administered prior to the first treatment cycle, i.e., prior to Cycle 1 Day 1. Thus, a treatment cycle, as used herein, refers to a 28-day treatment cycle. As used herein with respect to treatment cycles, “C1” refers to Cycle 1, “C2” refers to Cycle 2, “C3” refers to Cycle 3, and so on. Multiple cycles may also be described, e.g., “C3-6” refers to Cycles 3-6 (Cycles 3, 4, 5 and 6). A cycle number with a “+” symbol refers to that cycle and all subsequent cycles, e.g., “C5+” refers to from Cycle 5 and all subsequent cycles (i.e., C5, C6, C7, C8, C9, and so on).

As used herein “Q4W” means once every four weeks, “Q2W” (also referred to as “bi-weekly” or “biweekly”) means once every two weeks, and “QW” (also referred to as “weekly”) means once weekly. Q4W is sometimes referred to herein as “monthly” but technically refers to once every 4 weeks or once every 28 days (e.g., in 28-day cycles, a first treatment dose occurs on Day 1 of Cycle 1, a second treatment dose occurs on Day 1 of Cycle 2, etc.). Administration of a treatment dose once weekly (QW) is also referred to herein as a weekly dosing schedule; for example, a 28-day treatment cycle may have a weekly dosing schedule that comprises four doses one week apart from each other (e.g., on Days 1, 8, 15 and 22), or three doses one week apart from each other (e.g., on Days 8, 15 and 22), or two doses one week apart from each other (e.g., on Days 8 and 15). Administration of a treatment dose once every two weeks (Q2W) is also referred to herein as a bi-weekly dosing schedule. Administration of a treatment dose once every four weeks (Q4W) is also referred to herein as a monthly dosing schedule. Dosing regimens may be described herein in terms of the dose amount and frequency; for example, “C1:0.4 mg/kg QW” refers to administration of 0.4 mg/kg once per week in Cycle 1 of a therapeutically effective regimen, “C3-6:0.8 mg/kg Q2W” refers to administration of 0.8 mg/kg once every two weeks from Cycle 3 through Cycle 6, “C7+: 0.8 mg/kg Q4W” refers to administration of 0.8 mg/kg once every four weeks starting in Cycle 7, etc.

As used herein, a “GPRC5DxCD3 treatment cycle” refers to each treatment cycle in a therapeutically effective regimen in which at least one treatment dose of a GPRC5DxCD3 bispecific antibody is administered to the subject. In preferred embodiments, the first GPRC5DxCD3 treatment cycle in a therapeutically effective regimen is preceded by a step-up phase.

According to embodiments of the present invention, methods of treating multiple myeloma are effective in eliciting a clinical response in a subject as determined by 5 International Myeloma Working Group (IMWG) response criteria. According to particular embodiments, the methods of treatment are effective in eliciting a partial response, a very good partial response, a complete response or a stringent complete response, as determined by IMWG response criteria. As used herein, overall response rate (ORR) refers to the percentage of patients in a population that achieve a partial response (PR) or better, i.e., a partial response, very good partial response, complete response or stringent complete 10 response. IMWG criteria for response to Multiple Myeloma treatment are provided in Table 7 below.

TABLE 7

| Response | Response Criteria |
|---|---|
| sCR = stringent | CR as defined below, plus |
| complete response | Normal FLC ratio, and |
| | Absence of clonal PCs by immunohistochemistry, |
| | immunofluorescence^{a} or 2- to 4-color flow |
| | cytometry |
| CR = complete | Negative immunofixation on the serum and urine, |
| response | and |
| | Disappearance of any soft tissue plasmacytomas, |
| | and |
| | <5% PCs in bone marrow |
| VGPR = very good | Serum and urine M-component detectable by |
| partial response | immunofixation but not on electrophoresis, or |
| | ≥90% reduction in serum M-protein plus urine M- |
| | protein <100 mg/24 hours |
| PR = partial | ≥50% reduction of serum M-protein and reduction |
| response | in 24-hour urinary M-protein by ≥90% or to <200 |
| | mg/24 hours |
| | If the serum and urine M-protein are not |
| | measurable, a decrease of ≥50% in the difference |
| | between involved and uninvolved FLC levels is |
| | required in place of the M-protein criteria |
| | If serum and urine M-protein are not measurable, |
| | and serum free light assay is also not |
| | measurable, ≥50% reduction in bone marrow PCs |
| | is required in place of M-protein, |
| | provided baseline bone marrow plasma cell |
| | percentage was ≥30% |
| | In addition to the above criteria, if present at |
| | baseline, a ≥50% reduction in the size of soft tissue |
| | plasmacytomas is also required. |

CR = complete response; FLC = free light chain; IMWG = International Myeloma Working Group; M-protein = monoclonal paraprotein; MR = minimal response; PC = plasma cell; PD = progressive disease; PR = partial response; sCR = stringent complete response; SD = stable disease; VGPR = very good partial response
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
* Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.

IMWG criteria for response to Multiple Myeloma treatment are also described, for example, in Durie et al., Kumar et al. and Rajkumar et al., which are incorporated by reference herein: Durie BG, Harousseau J L, Miguel J S, et al. International uniform response criteria for multiple myeloma. Leukemia. 2006; 20 (9): 1467-1473; Kumar S, Paiva B, Anderson K C, et al. International Myeloma Working Group consensus criteria for response and minimal residual disease assessment in multiple myeloma. Lancet Oncol. 2016; 17 (8): e328-346; Rajkumar SV, Harousseau JL, Durie B, et al. Consensus recommendations for the uniform reporting of clinical Trials: report of the International Myeloma Workshop Consensus Panel 1. Blood. 2011; 117 (18): 4691-4695.

In certain embodiments, prior to switching to subcutaneous monthly (Q4W) dosing at a treatment dose amount of 0.8 mg/kg, talquetamab may be subcutaneously administered according to one or both of the following weight-based dosing schedules (QW and/or Q2W) shown in Tables 9 and 10, wherein mg/kg refers to mg of talquetamab per kg of the patient's body weight:

TABLE 9

| Phase | Day | Talquetamab Dose^{a} | |
|---|---|---|---|
| Step-up Phase | Day 1 | 0.01 | mg/kg |
| | Day 3^{b} | 0.06 | mg/kg |
| | Day 5^{b} | 0.4 | mg/kg |
| Treatment Phase | Once a week thereafter^{c} | 0.4 | mg/kg |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

TABLE 10

| Phase | Day | Talquetamab Dose^{a} | |
|---|---|---|---|
| Step-up Phase | Day 1 | 0.01 | mg/kg |
| | Day 3^{b} | 0.06 | mg/kg |
| | Day 5^{b} | 0.4 | mg/kg |
| | Day 7^{b} | 0.8 | mg/kg |
| Treatment Phase | Once every 2 weeks | 0.8 | mg/kg |
| | thereafter^{c} | | |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

According to certain embodiments, the dose frequency of a GPRC5DxCD3 bispecific antibody may be reduced from bi-weekly to monthly. For example, the dose frequency of a GPRC5DxCD3 bispecific antibody may be reduced from bi-weekly to monthly if the subject achieves a clinical response.

According to an embodiment, the dose frequency of a GPRC5DxCD3 bispecific antibody may be reduced after 4 treatment cycles from weekly to every other week if patients achieved a partial response and reduced further from every other week to monthly after 8 cycles if they achieved a very good partial response.

According to another embodiment, a dosing regimen of a GPRC5DxCD3 bispecific antibody may comprise 0.8 mg/kg biweekly treatment doses, followed by a switch to 0.8 mg/kg monthly at treatment Cycle 7, or a switch to 0.8 mg/kg monthly at treatment Cycle 5 if the subject achieves a complete response.

According to another embodiment, a dosing regimen of a GPRC5DxCD3 bispecific antibody may comprise subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

According to another embodiment, a dosing regimen of a GPRC5DxCD3 bispecific antibody may comprise subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

According to another embodiment, a dosing regimen of a GPRC5DxCD3 bispecific antibody may comprise subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7 or later, if the subject has achieved a partial response, a very good partial response, a complete response or a stringent complete response, as determined by IMWG response criteria.

Additional embodiments of the present invention are described below.

In certain embodiments, a method of treating multiple myeloma in a subject in need thereof comprises administering to the subject treatment doses of a GPRC5DxCD3 bispecific antibody on a monthly dosing schedule (Q4W).

In certain embodiments, the method comprises subcutaneously administering treatment doses of the GPRC5DxCD3 bispecific antibody on the monthly dosing schedule (Q4W).

In certain embodiments, the method comprises subcutaneously administering to the subject one or more step-up doses of the GPRC5DxCD3 bispecific antibody prior to administering the first treatment dose of the GPRC5DxCD3 bispecific antibody.

In certain embodiments, the method comprises subcutaneously administering the GPRC5DxCD3 bispecific antibody on the monthly dosing schedule (Q4W) at a treatment dose of from about 400 μg/kg to about 800 μg/kg.

In certain embodiments, the method comprises subcutaneously administering the GPRC5DxCD3 bispecific antibody on the monthly dosing schedule (Q4W) at a treatment dose of about 400 μg/kg.

In certain embodiments, the method comprises subcutaneously administering the GPRC5DxCD3 bispecific antibody on the monthly dosing schedule (Q4W) at a treatment dose of about 800 μg/kg.

In certain embodiments, the method comprises subcutaneously administering to the subject at least one treatment dose of the GPRC5DxCD3 bispecific antibody on a bi-weekly dosing schedule (Q2W) and subsequently administering to the subject treatment doses of the GPRC5DxCD3 bispecific antibody on the monthly dosing schedule (Q4W).

In certain embodiments, the method comprises subcutaneously administering to the subject at least one treatment dose of the GPRC5DxCD3 bispecific antibody on a weekly dosing schedule (QW) and then subcutaneously administering to the subject at least one treatment dose of the GPRC5DxCD3 bispecific antibody on a bi-weekly dosing schedule (Q2W) and then subsequently administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 6 treatment cycles, and starting at treatment cycle 7, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W), and if the patient has achieved a PR, VGPR, CR or sCR, as determined by

IMWG response criteria, then administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W), and if the patient has achieved a PR, VGPR, CR or sCR, as determined by

IMWG response criteria, then administering to the subject treatment doses of the GPRC5DxCD3 bispecific antibody on the monthly dosing schedule (Q4W) starting in treatment cycle 3, 4 or 5.

In certain embodiments, the method comprises subcutaneously administering to the subject treatment doses of the GPRC5DxCD3 bispecific antibody on a bi-weekly dosing schedule (Q2W) for 6 treatment cycles, and if the patient has achieved a VGPR, CR or sCR, as determined by IMWG response criteria, then starting at treatment cycle 7, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 7 treatment cycles, and starting at treatment cycle 8, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 7 treatment cycles, and if the patient has achieved a VGPR, CR or sCR, as determined by IMWG response criteria, then starting at treatment cycle 8, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 8 treatment cycles, and starting at treatment cycle 9, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) for 8 treatment cycles, and if the patient has achieved a VGPR, CR or sCR, as determined by IMWG response criteria, then starting at treatment cycle 9, administering to the subject treatment doses of the GPRC5DxCD3 bispecific antibody on the monthly dosing schedule (Q4W).

In certain embodiments, the method comprises subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 7 or treatment cycle 8.

In certain embodiments, the method comprises subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 5 if the subject has achieved a complete response or a stringent complete response, as determined by IMWG response criteria.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7 or later, if the subject has achieved a partial response, a very good partial response, a complete response or a stringent complete response, as determined by IMWG response criteria.

In certain embodiments, each treatment dose amount of the GPRC5DxCD3 bispecific antibody administered on a weekly dosing schedule (QW), a bi-weekly dosing schedule (Q2W) or the monthly dosing schedule (Q4W), is the same.

In certain embodiments, the GPRC5DxCD3 bispecific antibody is administered on the bi-weekly dosing schedule (Q2W) and the monthly dosing schedule (Q4W), at a treatment dose amount of 800 μg/kg.

In certain embodiments, the method comprises subcutaneously administering 2 or 3 step-up doses of the GPRC5DxCD3 bispecific antibody prior to subcutaneously administering the first treatment dose.

In certain embodiments, the method comprises subcutaneously administering step-up doses of 10 μg/kg and 60 μg/kg of the GPRC5DxCD3 bispecific antibody prior to subcutaneously administering a treatment dose.

In certain embodiments, the method comprises subcutaneously administering step-up doses of 10 μg/kg and 60 μg/kg and 400 μg/kg of the GPRC5DxCD3 bispecific antibody prior to subcutaneously administering a treatment dose.

In certain embodiments, the method comprises subcutaneously administering step-up doses of the GPRC5DxCD3 bispecific antibody 2-4 days apart from each other.

In certain embodiments, the subject achieves a clinical response that is a PR, or a VGPR, or a CR or a sCR.

In certain embodiments, the subject achieves a clinical response that is a CR or a sCR.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

In certain embodiments, the method comprises subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 or 6 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, if the subject has achieved a partial response, a very good partial response, a complete response or a stringent complete response, as determined by IMWG response criteria. In certain embodiments, the method comprises subcutaneously administering 2 or 3 step-up doses of the GPRC5DxCD3 bispecific antibody prior to subcutaneously administering the first treatment dose.

In certain embodiments, the method comprises subcutaneously administering step-up doses of 10 μg/kg and 60 μg/kg of the GPRC5DxCD3 bispecific antibody prior to subcutaneously administering a treatment dose.

In certain embodiments, the method comprises subcutaneously administering step-up doses of 10 μg/kg and 60 μg/kg and 400 μg/kg of the GPRC5DxCD3 bispecific antibody prior to subcutaneously administering a treatment dose.

In certain embodiments, the method comprises subcutaneously administering step-up doses of the GPRC5DxCD3 bispecific antibody 2-4 days apart from each other.

In certain embodiments, the method comprises treating the subject according to a therapeutically effective regimen that comprises sequential 28-day GPRC5DxCD3 treatment cycles, wherein: one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W).

In certain embodiments, the method comprises treating the subject according to a therapeutically effective regimen that comprises sequential 28-day GPRC5DxCD3 treatment cycles, wherein: one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

In certain embodiments, the method comprises treating the subject according to a therapeutically effective regimen that comprises sequential 28-day GPRC5DxCD3 treatment cycles, wherein: one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, if the subject has achieved a partial response, a very good partial response, a complete response or a stringent complete response, as determined by IMWG response criteria.

In certain embodiments, each treatment dose of the GPRC5DxCD3 bispecific antibody administered on the bi-weekly dosing schedule (Q2W) and the monthly dosing schedule (Q4W) is 800 μg/kg.

In certain embodiments, Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W).

In certain embodiments, the dosing regimen comprises a step-up phase and then treatment doses of the GPRC5DxCD3 bispecific antibody are subcutaneously administered to the subject on the monthly dosing schedule (Q4W) starting from the treatment cycle immediately following the step-up phase (regardless of clinical response, or lack of clinical response, in the subject). In certain embodiments, Cycle 1 of the regimen comprises the step-up phase, and the treatment doses of the GPRC5DxCD3 bispecific antibody are subcutaneously administered to the subject on the monthly dosing schedule (Q4W) starting from Cycle 2 (e.g., 0.8 mg/kg on Day 1 of each cycle starting in Cycle 2). In certain embodiments, the step-up phase comprises 2 or 3 step-up doses, preferably 2-4 days apart from each other, followed by at least one treatment dose of 0.8 mg/kg dose (e.g., one treatment dose at Cycle 1 Day 15). In certain embodiments, the step-up phase comprises 3 step-up doses (0.01, 0.06, and 0.4 mg/kg), e.g., on Cycle 1 Days 2, 4, and 8, followed by a treatment dose of 0.8 mg/kg dose, e.g., at Cycle 1 Day 15.

In certain embodiments, Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment

Cycle 7, regardless of clinical response in the subject.

In certain embodiments, 1-3 step-up doses of the GPRC5DxCD3 bispecific antibody are administered to the subject during the step-up phase.

In certain embodiments, two step-up doses of the GPRC5DxCD3 bispecific antibody are administered to the subject during the step-up phase.

In certain embodiments, three step-up doses of the GPRC5DxCD3 bispecific antibody are administered to the subject during the step-up phase.

In certain embodiments, the step-up phase comprises a first step-up dose of 0.01 mg/kg, a second step-up dose of 0.06 mg/kg, and a third step-up dose of 0.4 mg/kg.

In certain embodiments, one or more treatment doses of the GPRC5DxCD3 bispecific antibody are subcutaneously administered to the subject during the step-up phase, in addition to the one or more step-up doses (e.g., on a weekly dosing schedule).

In certain embodiments, one or two treatment doses of the GPRC5DxCD3 bispecific antibody are subcutaneously administered to the subject during the step-up phase, in addition to the one or more step-up doses (e.g., on a weekly dosing schedule).

In certain embodiments, Cycle 1 of the regimen comprises a first step-up dose of 0.01 mg/kg, a second step-up dose of 0.06 mg/kg, a third step-up dose of 0.4 mg/kg, and a treatment dose of 0.8 mg/kg.

In certain embodiments, Cycle 1 of the regimen comprises a first step-up dose of 0.01 mg/kg, a second step-up dose of 0.06 mg/kg 2-4 days after the first step-up dose, a third step-up dose of 0.4 mg/kg 4-7 days after the second step-up dose, and a treatment dose of 0.8 mg/kg 5-9 days after the third step-up dose.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15.

In certain embodiments, each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1.

In certain embodiments, either: (i) each of Cycles 2-6 of the regimen comprises a

Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and each of Cycles 2-6 of the regimen comprises a Q2W treatment dose of 0.8 mg/kg on Day 1 and Day 15; and each of Cycles 7+ comprises a Q4W treatment dose of 0.8 mg/kg on Day 1.

In certain embodiments, Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and either: (i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

A non-limiting example of an exemplary dosing regimen of the present invention is provided in Table 11.

TABLE 11
Study

| Treatment | Dose Schedule (each cycle = 28 days) | |
|---|---|---|
| Talquetamab | Step-up dose 1: 0.01 mg/kg; Step-up dose 2: 0.06 mg/kg; | |
| (subcutaneous) | | Step-up dose 3: 0.4 mg/kg; Each treatment dose: |
| | | 0.8 mg/kg |
| | C1: | Step-up dose 1: Day 2; Step-up dose 2: Day 3 |
| | | or 4 (or up to Day 6); Step-up dose 3: Day 7 or 8 |
| | | (or up to Day 10); Treatment dose: Day 15 (or up |
| | | to Day 17) |
| | C2-4: | Treatment dose on Days 1 and 15 |
| | C5-6: | Treatment dose on Days 1 and 15 or if confirmed |
| | | VGPR, may change to Q4W dosing (Day 1 only) |
| | C7+: | Treatment dose on Day 1 only (Q4W) |

Another non-limiting example of an exemplary dosing regimen of the present invention is provided in Table 12.

TABLE 12
Study

| Treatment | Dose Schedule (each cycle = 28 days) | |
|---|---|---|
| Talquetamab | C1: | 3 step-up doses (0.01, 0.06, and 0.4 mg/kg) on |
| (subcutaneous) | | Days 1, 3, and 8 followed by treatment dose |
| | | (0.8 mg/kg) on Day 15 |
| | C2-6*: | 0.8 mg/kg Q2W on Days 1 and 15 |
| | C7+: | 0.8 mg/kg Q4W on Day 1 |

*Subjects may switch to 0.8 mg/kg Q4W starting at Cycle 5 Day 1 with response of VGPR or better.

Another non-limiting example of an exemplary dosing regimen of the present invention is provided below, in which talquetamab is administered in 28-day treatment cycles:
In Cycle 1, the following doses of subcutaneous talquetamab are administered:
   Talquetamab step-up dose 1 (SU1): 0.01 mg/kg, administered on Day 1;
   Talquetamab step-up dose 2 (SU2): 0.06 mg/kg, administered >2d after SU1;
   Talquetamab treatment dose (sometimes referred to as step-up dose 3): 0.4 mg/kg, administered ≥2d after SU2;
   Talquetamab treatment dose: 0.8 mg/kg, administered >2d after first treatment dose (step-up dose 3) of 0.4 mg/kg, between Days 7-15;
In subsequent treatment cycles following Cycle 1, the subcutaneous Talquetamab treatment doses of 0.8 mg/kg are administered as follows:
   Cycle 2-Cycle 4 (C2-C4), Talquetamab SC is administered bi-weekly (Q2W), i.e., 14 days (±3d) after prior treatment dose;
   From Cycle 5 (C5), if confirmed VGPR or better, schedule can change to Q4W dosing (Day 1 of each 28-day cycle only), with the change to Q4W occurring on Cycle 5 or Cycle 6 Day 1 (±3d);
   At Cycle 7 Day 1 (±3d), if confirmed PR or better, schedule must change to Q4W dosing;
   At Cycle 7 Day 1 (±3d), if not in confirmed PR or better, continue with Q2W dosing until confirmed PR or better is achieved.

Another non-limiting example of an exemplary dosing regimen of the present invention is provided in Table 13.

TABLE 13
Study

| Treatment | Dose Schedule (each cycle = 28 days) | |
|---|---|---|
| Talquetamab | C1: | 3 step-up doses (0.01, 0.06, and 0.4 mg/kg) on Days |
| SC | | 2, 4, and 8 followed by treatment dose (0.8 mg/kg) |
| | | on Day 15 |
| | C2+: | Q4W treatment doses (0.8 mg/kg) on Day 1 |

In certain embodiments, a dosing regimen comprising administration of the GPRC5DxCD3 bispecific antibody on the monthly (Q4W) dosing schedule improves (i) the clinical response rate (e.g., ORR and/or PFS) and/or (ii) the rate and/or severity of adverse events in a population of subjects compared to a reference population of subjects that receives the GPRC5DxCD3 bispecific antibody on a weekly (QW) and/or bi-weekly (Q2W) dosing schedule and not a monthly (Q4W) dosing schedule.

In certain embodiments, a dosing regimen comprising the monthly (Q4W) dosing schedule achieves a lower rate and/or lesser duration of adverse events than a dosing regimen comprising a weekly (QW) and/or bi-weekly (Q2W) dosing schedule and not a monthly (Q4W) dosing schedule.

In certain embodiments, the adverse events comprise one or more of oral toxicity (e.g., ageusia, dysgeusia, dry mouth, and/or dysphagia), and/or nail toxicity (e.g., nail disorder), and/or skin toxicity (e.g., dry skin, skin exfoliation, and/or pruritus)

In certain embodiments, a dosing regimen comprising the monthly (Q4W) dosing schedule achieves a stronger and/or longer duration of clinical response than a dosing regimen comprising a weekly (QW) and/or bi-weekly (Q2W) dosing schedule and not a monthly (Q4W) dosing schedule.

### Exemplary Embodiments

Provided below are enumerated embodiments of the present invention. These embodiments are illustrative only and do not limit the scope of the present disclosure or of the claims attached hereto.
1. A method of treating multiple myeloma in a subject in need thereof, comprising administering to the subject a therapeutically effective amount of a GPRC5DxCD3 bispecific antibody on a monthly (Q4W) dosing schedule.
2. The method of embodiment 1, wherein the GPRC5DxCD3 bispecific antibody comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
3. The method of embodiment 1 or 2, wherein the GPRC5DxCD3 bispecific antibody comprises a GPRC5D binding domain comprising a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
4. The method of any of embodiments 1-3, wherein the GPRC5DxCD3 bispecific antibody is an IgG1, an IgG2, an IgG3 or an IgG4 isotype.
5. The method of any of embodiments 1-4, wherein the GPRC5DxCD3 bispecific antibody is an IgG4 isotype.
6. The method of any of embodiments 1-5, wherein the GPRC5DxCD3 bispecific antibody comprises one or more substitutions in its Fc region.
7. The method of any of embodiments 1-6, wherein the GPRC5DxCD3 bispecific antibody is an IgG4 isotype and comprises S228P, F234A and L235A substitutions in its Fc region (according to EU numbering).
8. The method of any of embodiments 1-7, wherein the GPRC5DxCD3 bispecific antibody is an IgG4 isotype and comprises S228P, F234A, L235A F405L and R409K substitutions in its Fc region (according to EU numbering).
9. The method of any of embodiments 1-8, wherein the Fc region of the GPRC5D binding arm comprises S228P, F234A and L235A substitutions in its Fc region (according to EU numbering).
10. The method of any of embodiments 1-9, wherein the Fc region of the CD3 binding arm comprises S228P, F234A, L235A, F405L, and R409K substitutions in its Fc region (according to EU numbering).
11. The method of any of embodiments 1-10, wherein the GPRC5DxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
12. The method of any of embodiments 1-10, wherein the GPRC5DxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.
13. The method of any of embodiments 1-10, wherein the GPRC5DxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.
14. The method of any of embodiments 1-10, wherein the GPRC5DxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.
15. The method of any of embodiments 1-10, wherein the GPRC5DxCD3 bispecific antibody is talquetamab.
16. The method of any of embodiments 1-15, wherein the subject has relapsed or refractory multiple myeloma myeloma.
17. The method of any of embodiments 1-16, wherein the subject has received at least three prior lines of therapy.
18. The method of any of embodiments 1-16, wherein the subject has received at least four prior lines of therapy.
19. The method of any of embodiments 1-16, wherein the subject has received at least five prior lines of therapy (penta-drug exposed).
20. The method of any of embodiments 1-16, wherein the subject has received at least three prior lines of therapy, including a proteasome inhibitor, an immunomodulatory agent and an anti-CD38 monoclonal antibody.
21. The method of any of embodiments 1-16, wherein the subject has received at least four prior lines of therapy, including a proteasome inhibitor, an immunomodulatory agent and an anti-CD38 monoclonal antibody.
22. The method of any of embodiments 1-21 comprising subcutaneously administering treatment doses of the GPRC5DxCD3 bispecific antibody on the monthly dosing schedule (Q4W).
23. The method of any of embodiments 1-22 comprising subcutaneously administering to the subject one or more step-up doses of the GPRC5DxCD3 bispecific antibody prior to administering the first treatment dose of the GPRC5DxCD3 bispecific antibody.
24. The method of any of embodiments 1-23 comprising subcutaneously administering the GPRC5DxCD3 bispecific antibody on the monthly dosing schedule (Q4W) at a treatment dose of from about 400 μg/kg to about 800 μg/kg.
25. The method of any of embodiments 1-23 comprising subcutaneously administering the GPRC5DxCD3 bispecific antibody on the monthly dosing schedule (Q4W) at a treatment dose of about 400 μg/kg.
26. The method of any of embodiments 1-23 comprising subcutaneously administering the GPRC5DxCD3 bispecific antibody on the monthly dosing schedule (Q4W) at a treatment dose of about 800 μg/kg.
27. The method of any of embodiments 1-26 comprising subcutaneously administering to the subject at least one treatment dose of the GPRC5DxCD3 bispecific antibody on a bi-weekly dosing schedule (Q2W) and subsequently administering to the subject treatment doses of the GPRC5DxCD3 bispecific antibody on the monthly dosing schedule (Q4W).
28. The method of any of embodiments 1-27 comprising subcutaneously administering to the subject at least one treatment dose of the GPRC5DxCD3 bispecific antibody on a weekly dosing schedule (QW) and then subcutaneously administering to the subject at least one treatment dose of the GPRC5DxCD3 bispecific antibody on a bi-weekly dosing schedule (Q2W) and then subsequently administering to the subject treatment doses of the GPRC5DxCD3 bispecific antibody on the monthly dosing schedule (Q4W).
29. The method of any of embodiments 1-28 comprising subcutaneously administering to the subject treatment doses of the GPRC5DxCD3 bispecific antibody on a bi-weekly dosing schedule (Q2W) for 6 treatment cycles, and starting at treatment cycle 7, administering to the subject treatment doses of the GPRC5DxCD3 bispecific antibody on the monthly dosing schedule (Q4W).
30. The method of any of embodiments 1-28 comprising subcutaneously administering to the subject treatment doses of the GPRC5DxCD3 bispecific antibody on a bi-weekly dosing schedule (Q2W) for 6 treatment cycles, and if the patient has achieved a VGPR, CR or sCR, as determined by IMWG response criteria, then starting at treatment cycle 7, administering to the subject treatment doses of the GPRC5DxCD3 bispecific antibody on the monthly dosing schedule (Q4W).
31. The method of any of embodiments 1-28 comprising subcutaneously administering to the subject treatment doses of the GPRC5DxCD3 bispecific antibody on a bi-weekly dosing schedule (Q2W) for 7 treatment cycles, and starting at treatment cycle 8, administering to the subject treatment doses of the GPRC5DxCD3 bispecific antibody on the monthly dosing schedule (Q4W).
32. The method of any of embodiments 1-28 comprising subcutaneously administering to the subject treatment doses of the GPRC5DxCD3 bispecific antibody on a bi-weekly dosing schedule (Q2W) for 7 treatment cycles, and if the patient has achieved a VGPR, CR or sCR, as determined by IMWG response criteria, then starting at treatment cycle 8, administering to the subject treatment doses of the GPRC5DxCD3 bispecific antibody on the monthly dosing schedule (Q4W).
33. The method of any of embodiments 1-28 comprising subcutaneously administering to the subject treatment doses of the GPRC5DxCD3 bispecific antibody on a bi-weekly dosing schedule (Q2W) for 8 treatment cycles, and starting at treatment cycle 9, administering to the subject treatment doses of the GPRC5DxCD3 bispecific antibody on the monthly dosing schedule (Q4W).
34. The method of any of embodiments 1-28 comprising subcutaneously administering to the subject treatment doses of the GPRC5DxCD3 bispecific antibody on a bi-weekly dosing schedule (Q2W) for 8 treatment cycles, and if the patient has achieved a VGPR, CR or sCR, as determined by IMWG response criteria, then starting at treatment cycle 9, administering to the subject treatment doses of the GPRC5DxCD3 bispecific antibody on the monthly dosing schedule (Q4W).
35. The method of any of embodiments 1-27, comprising subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 7 or treatment cycle 8.
36. The method of any of embodiments 1-27, comprising subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 5 if the subject has achieved a complete response or a stringent complete response, as determined by IMWG response criteria.
37. The method of any of embodiments 27-36 wherein each treatment dose amount of the GPRC5DxCD3 bispecific antibody administered on a weekly dosing schedule (QW), a bi-weekly dosing schedule (Q2W) or the monthly dosing schedule (Q4W), is the same.
38. The method of any of embodiments 27-36 wherein the GPRC5DxCD3 bispecific antibody is administered on the bi-weekly dosing schedule (Q2W) and the monthly dosing schedule (Q4W), at a treatment dose amount of 800 μg/kg.
39. The method of any of embodiments 1-38, comprising subcutaneously administering 2 or 3 step-up doses of the GPRC5DxCD3 bispecific antibody prior to subcutaneously administering the first treatment dose.
40. The method of any of embodiments 1-38, comprising subcutaneously administering step-up doses of 10 μg/kg and 60 μg/kg of the GPRC5DxCD3 bispecific antibody prior to subcutaneously administering a treatment dose.
41. The method of any of embodiments 1-38, comprising subcutaneously administering step-up doses of 10 μg/kg and 60 μg/kg and 400 μg/kg of the GPRC5DxCD3 bispecific antibody prior to subcutaneously administering a treatment dose.
42. The method of any of embodiments 1-41, comprising subcutaneously administering step-up doses of the GPRC5DxCD3 bispecific antibody 2-4 days apart from each other.
43. The method of any of embodiments 1-42, wherein the subject achieves a clinical response that is a PR, or a VGPR, or a CR or a sCR.
44. The method of any of embodiments 1-42, wherein the subject achieves a clinical response that is a CR or a sCR.
45. The method of any of embodiments 1-27, comprising subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.
46. The method of embodiment 45 wherein the GPRC5DxCD3 bispecific antibody is administered on the bi-weekly dosing schedule (Q2W) and the monthly dosing schedule (Q4W), at a treatment dose amount of 800 μg/kg.
47. The method of any of embodiments 45-46, comprising subcutaneously administering 2 or 3 step-up doses of the GPRC5DxCD3 bispecific antibody prior to subcutaneously administering the first treatment dose.
48. The method of any of embodiments 45-47, comprising subcutaneously administering step-up doses of 10 μg/kg and 60 μg/kg of the GPRC5DxCD3 bispecific antibody prior to subcutaneously administering a treatment dose.
49. The method of any of embodiments 45-48, comprising subcutaneously administering step-up doses of 10 μg/kg and 60 μg/kg and 400 μg/kg of the GPRC5DxCD3 bispecific antibody prior to subcutaneously administering a treatment dose.
50. The method of any of embodiments 45-49, comprising subcutaneously administering step-up doses of the GPRC5DxCD3 bispecific antibody 2-4 days apart from each other.
51. The method of any of embodiments 45-50, wherein the subject achieves a clinical response that is a PR, or a VGPR, or a CR or a sCR.
52. The method of any of embodiments 45-51, wherein the subject achieves a clinical response that is a CR or a sCR.
53. The method of any of embodiments 1-52, comprising treating the subject according to a therapeutically effective regimen that comprises sequential 28-day GPRC5DxCD3 treatment cycles, wherein:
one or more step-up doses of the GPRC5DxCD3 bispecific antibody are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W).
54. The method of any of embodiments 1-53, comprising treating the subject according to a therapeutically effective regimen that comprises sequential 28-day GPRC5DxCD3 treatment cycles, wherein:
one or more step-up doses of the GPRC5DxCD3 bispecific antibody are subcutaneously administered to the subject during a step-up phase, and
treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.
55. The method of embodiment 53 or 54, wherein each treatment dose of the GPRC5DxCD3 bispecific antibody administered on the bi-weekly dosing schedule (Q2W) and the monthly dosing schedule (Q4W) is 800 μg/kg.
56. The method of any of embodiments 53-55, wherein Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W).
57. The method of any of embodiments 53-56, wherein Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.
58. The method of any of embodiments 53-57, wherein 1-3 step-up doses of the GPRC5DxCD3 bispecific antibody are administered to the subject during the step-up phase.
59. The method of any of embodiments 53-58, wherein 2 step-up doses of the GPRC5DxCD3 bispecific antibody are administered to the subject during the step-up phase.
60. The method of any of embodiments 53-58, wherein 3 step-up doses of the GPRC5DxCD3 bispecific antibody are administered to the subject during the step-up phase.
61. The method of any of embodiments 53-60, wherein the step-up phase comprises a first step-up dose of 0.01 mg/kg, a second step-up dose of 0.06 mg/kg, and a third step-up dose of 0.4 mg/kg.
62. The method of any of embodiments 53-61, wherein one or more treatment doses of the GPRC5DxCD3 bispecific antibody are subcutaneously administered to the subject during the step-up phase, in addition to the one or more step-up doses (e.g., on a weekly dosing schedule).
63. The method of any of embodiments 53-61, wherein one or two treatment doses of the GPRC5DxCD3 bispecific antibody are subcutaneously administered to the subject during the step-up phase, in addition to the one or more step-up doses (e.g., on a weekly dosing schedule).
64. The method of any of embodiments 53-63, wherein Cycle 1 of the regimen comprises a first step-up dose of 0.01 mg/kg, a second step-up dose of 0.06 mg/kg, a third step-up dose of 0.4 mg/kg, and a treatment dose of 0.8 mg/kg.
65. The method of any of embodiments 53-64, wherein Cycle 1 of the regimen comprises a first step-up dose of 0.01 mg/kg, a second step-up dose of 0.06 mg/kg 2-4 days after the first step-up dose, a third step-up dose of 0.4 mg/kg 4-7 days (or 5-7 days) after the second step-up dose, and a treatment dose of 0.8 mg/kg 5-9 days (or 7-9 days) after the third step-up dose.
66. The method of any of embodiments 53-65, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15.
67. The method of any of embodiments 53-66, wherein each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1.
68. The method of any of embodiments 53-66, wherein either: (i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.
69. The method of any of embodiments 53-68, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and each of Cycles 2-6 of the regimen comprises a Q2W treatment dose of 0.8 mg/kg on Day 1 and Day 15; and each of Cycles 7+ comprises a Q4W treatment dose of 0.8 mg/kg on Day 1.
70. The method of any of embodiments 53-68, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and either:
(i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or
(ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.
71. The method of any of embodiments 1-53, comprising treating the subject according to a therapeutically effective regimen that comprises sequential 28-day GPRC5DxCD3 treatment cycles, wherein:
one or more step-up doses of the GPRC5DxCD3 bispecific antibody (e.g., talquetamab) are subcutaneously administered to the subject during a step-up phase, and treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 or 6 if the subject has achieved a very good partial response, a complete response or a stringent complete response, as determined by IMWG response criteria; or (ii) starting at treatment Cycle 7 or later, if the subject has achieved a partial response, a very good partial response, a complete response or a stringent complete response, as determined by IMWG response criteria, wherein each treatment dose of the GPRC5DxCD3 bispecific antibody administered on the bi-weekly dosing schedule (Q2W) and the monthly dosing schedule (Q4W) is 0.8 mg/kg.
72. The method of embodiment 71, wherein the step-up phase comprises administering 2 or 3 step-up doses of the GPRC5DxCD3 bispecific antibody, preferably 2-4 days apart from each other.
73. The method of embodiment 71, wherein the step-up phase comprises administering 2 or 3 step-up doses of the GPRC5DxCD3 bispecific antibody and one treatment dose of 0.8 mg/kg.
74. The method of embodiment 71, wherein the step-up phase comprises administering 3 step-up doses of the GPRC5DxCD3 bispecific antibody (e.g., 0.01 mg/kg, 0.06 mg/kg, and 0.4 mg/kg) and one treatment dose of 0.8 mg/kg.
75. The method of any of embodiments 71-74, wherein Cycle 1 of the regimen comprises the step-up phase, and the bi-weekly dosing schedule (Q2W) starts in Cycle 2.
76. The method of any of embodiments 1-53, comprising treating the subject according to a therapeutically effective regimen that comprises sequential 28-day GPRC5DxCD3 treatment cycles, wherein: one or more step-up doses of the GPRC5DxCD3 bispecific antibody (e.g., talquetamab) are subcutaneously administered to the subject during a step-up phase, and then treatment doses of the GPRC5DxCD3 bispecific antibody are subcutaneously administered to the subject on the monthly dosing schedule (Q4W) starting from the treatment cycle immediately following the step-up phase (regardless of clinical response, or lack of clinical response, in the subject),
wherein each treatment dose of the GPRC5DxCD3 bispecific antibody administered on the bi-weekly dosing schedule (Q2W) and the monthly dosing schedule (Q4W) is 0.8 mg/kg.
77. The method of embodiment 76, wherein the step-up phase comprises administering 2 or 3 step-up doses of the GPRC5DxCD3 bispecific antibody, preferably 2-4 days apart from each other.
78. The method of embodiment 76, wherein the step-up phase comprises administering 2 or 3 step-up doses of the GPRC5DxCD3 bispecific antibody and one treatment dose of 0.8 mg/kg.
79. The method of embodiment 76, wherein the step-up phase comprises administering 3 step-up doses of the GPRC5DxCD3 bispecific antibody (e.g., 0.01 mg/kg, 0.06 mg/kg, and 0.4 mg/kg) and one treatment dose of 0.8 mg/kg.
80. The method of any of embodiments 76-79, wherein Cycle 1 of the regimen comprises the step-up phase, and the monthly dosing schedule (Q4W) starts in Cycle 2.
81. The method of any of embodiments 1-80, wherein the GPRC5DxCD3 bispecific antibody (e.g., talquetamab) is administered as a monotherapy.
82. The method of any of embodiments 1-80, wherein the GPRC5DxCD3 bispecific antibody (e.g., talquetamab) is administered as part of a combination therapy with one or more additional anti-myeloma agents.
83. The method of any of embodiments 1-82, wherein a dosing regimen comprising the monthly (Q4W) dosing schedule achieves a lower rate and/or lesser duration of adverse events than a dosing regimen comprising a weekly (QW) and/or bi-weekly (Q2W) dosing schedule and not a monthly (Q4W) dosing schedule.
84. The method of embodiment 83, wherein the adverse events comprise one or more of oral toxicity (e.g., ageusia, dysgeusia, dry mouth, and/or dysphagia), and/or nail toxicity (e.g., nail disorder), and/or skin toxicity (e.g., dry skin, skin exfoliation, and/or pruritus)
85. The method of any of embodiments 1-84, wherein a dosing regimen comprising the monthly (Q4W) dosing schedule achieves a stronger and/or longer duration of clinical response than a dosing regimen comprising a weekly (QW) and/or bi-weekly (Q2W) dosing schedule and not a monthly (Q4W) dosing schedule.
86. The method of any of embodiments 1-85, wherein a dosing regimen comprising the monthly (Q4W) dosing schedule achieves a higher overall response rate (ORR) than a dosing regimen comprising a weekly (QW) and/or bi-weekly (Q2W) dosing schedule and not a monthly (Q4W) dosing schedule.
87. The method of any of embodiments 1-86, wherein a dosing regimen comprising the monthly (Q4W) dosing schedule achieves longer progression-free survival (PFS) than a dosing regimen comprising a weekly (QW) and/or bi-weekly (Q2W) dosing schedule and not a monthly (Q4W) dosing schedule.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### Examples

The following examples are provided to further describe some of the embodiments disclosed herein. The examples are intended to illustrate, not to limit, the disclosed embodiments.

### Example 1: Efficacy and Safety of Less Frequent/Lower Intensity Dosing of Talquetamab in Patients With Relapsed/Refractory Multiple Myeloma: Results From the Phase 1/2 MonumenTAL-1 Study

### Antibodies

Anti-GPRC5D/anti-CD3 antibody talquetamab (also called Tal) was made by Janssen Pharmaceuticals. Talquetamab comprises a GPRC5D binding arm and a CD3 binding arm, the amino acid sequences of which are shown in Table 5 and Table 6, respectively.

TABLE 5
Sequences of GPRC5D binding arm

| | | | SEQ ID |
|---|---|---|---|
| | Region | Sequence | NO: |
| GPRC5D | HCDR1 | GYTMN | 4 |
| binding | HCDR2 | LINPYNSDTNYAQKLQG | 5 |
| arm | HCDR3 | VALRVALDY | 6 |
| | LCDR1 | KASQNVATHVG | 7 |
| | LCDR2 | SASYRYS | 8 |
| | LCDR3 | QQYNRYPYT | 9 |
| | VH | QVQLVQSGAEVKKPGASVKVSCKASGYSF | 10 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLV | |
| | | TVSS | |
| | VL | DIQMTQSPSSLSASVGDRVTITCKASQNVA | 11 |
| | | THVGWYQQKPGKAPKRLIYSASYRYSGVP | |
| | | SRFSGSGSGTEFTLTISNLQPEDFATYYCQQ | |
| | | YNRYPYTFGQGTKLEIK | |
| | HC | QVQLVQSGAEVKKPGASVKVSCKASGYSF | 12 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLV | |
| | | TVSSASTKGPSVFPLAPCSRSTSESTAALGC | |
| | | LVKDYFPEPVTVSWNSGALTSGVHTFPAV | |
| | | LQSSGLYSLSSVVTVPSSSLGTKTYTCNVD | |
| | | HKPSNTKVDKRVESKYGPPCPPCPAPEAAG | |
| | | GPSVFLFPPKPKDTLMISRTPEVTCVVVDV | |
| | | SQEDPEVQFNWYVDGVEVHNAKTKPREE | |
| | | QFNSTYRVVSVLTVLHQDWLNGKEYKCK | |
| | | VSNKGLPSSIEKTISKAKGQPREPQVYTLPP | |
| | | SQEEMTKNQVSLTCLVKGFYPSDIAVEWE | |
| | | SNGQPENNYKTTPPVLDSDGSFFLYSRLTV | |
| | | DKSRWQEGNVFSCSVMHEALHNHYTQKS | |
| | | LSLSLGK | |
| | LC | DIQMTQSPSSLSASVGDRVTITCKASQNVA | 13 |
| | | THVGWYQQKPGKAPKRLIYSASYRYSGVP | |
| | | SRFSGSGSGTEFTLTISNLQPEDFATYYCQQ | |
| | | YNRYPYTFGQGTKLEIKRTVAAPSVFIFPPS | |
| | | DEQLKSGTASVVCLLNNFYPREAKVQWK | |
| | | VDNALQSGNSQESVTEQDSKDSTYSLSSTL | |
| | | TLSKADYEKHKVYACEVTHQGLSSPVTKS | |
| | | FNRGEC | |

TABLE 6
Sequences of CD3 binding arm

| | | | SEQ |
|---|---|---|---|
| | Region | Sequence | ID NO: |
| CD3 | HCDR1 | TYAMN | 14 |
| binding | HCDR2 | RIRSKYNNYATYYAASVKG | 15 |
| arm | HCDR3 | HGNFGNSYVSWFAY | 16 |
| | LCDR1 | RSSTGAVTTSNYAN | 17 |
| | LCDR2 | GTNKRAP | 18 |
| | LCDR3 | ALWYSNLWV | 19 |
| | VH | EVQLVESGGGLVQPGGSLRLSCAASGFTFNT | 20 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSS | |
| | VL | QTVVTQEPSLTVSPGGTVTLTCRSSTGAVTT | 21 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQP | |
| | HC | EVQLVESGGGLVQPGGSLRLSCAASGFTFNT | 22 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSSASTKGPSVFPLAPCSRSTSESTAALGC | |
| | | LVKDYFPEPVTVSWNSGALTSGVHTFPAVL | |
| | | QSSGLYSLSSVVTVPSSSLGTKTYTCNVDHK | |
| | | PSNTKVDKRVESKYGPPCPPCPAPEAAGGPS | |
| | | VFLFPPKPKDTLMISRTPEVTCVVVDVSQED | |
| | | PEVQFNWYVDGVEVHNAKTKPREEQFNSTY | |
| | | RVVSVLTVLHQDWLNGKEYKCKVSNKGLPS | |
| | | SIEKTISKAKGQPREPQVYTLPPSQEEMTKNQ | |
| | | VSLTCLVKGFYPSDIAVEWESNGQPENNYKT | |
| | | TPPVLDSDGSFLLYSKLTVDKSRWQEGNVFS | |
| | | CSVMHEALHNHYTQKSLSLSLGK | |
| | LC | QTVVTQEPSLTVSPGGTVTLTCRSSTGAVTT | 23 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQPKAAPSVTLFP | |
| | | PSSEELQANKATLVCLISDFYPGAVTVAWKA | |
| | | DSSPVKAGVETTTPSKQSNNKYAASSYLSLT | |
| | | PEQWKSHRSYSCQVTHEGSTVEKTVAPTECS | |

### BACKGROUND

Talquetamab (tal) is an off-the-shelf, T-cell redirecting bispecific antibody targeting G protein-coupled receptor family C group 5 member D (GPRC5D) and CD3. Results from 5 the phase 1/2 MonumenTAL-1 (NCT03399799/NCT04634552) trial showed overall response rates (ORRs) of >71% and a manageable safety profile with the recommended phase 2 doses (RP2Ds) of subcutaneous tal (0.4 mg/kg weekly [QW] or 0.8 mg/kg every other week [Q2W]) in patients with relapsed/refractory multiple myeloma (RRMM). The impact of reducing dose intensity with bispecifics on safety and efficacy is an area of clinical interest. This example reports safety and efficacy in patients from MonumenTAL-1 who switched to less frequent or reduced dosing with Tal.

### Methods

In phase 1, patients were intolerant to or progressed on established therapies and had an Eastern Cooperative Oncology Group performance status (ECOG PS) of 0 or 1. In phase 2, patients had received >3 prior lines of therapy, including ≥1 proteasome inhibitor, ≥1 immunomodulatory drug, and ≥1 anti-CD38 monoclonal antibody, and had an ECOG PS of 0-2. Phase 1 included 2 prospectively designed cohorts: (A) a reduced dosing cohort in which patients treated with tal 0.8 mg/kg Q2W were permitted to switch to 0.4 mg/kg Q2W at the next cycle following a confirmed partial response or better (≥PR), and (B) a less frequent dosing cohort in which patients treated with tal 0.8 mg/kg Q2W were permitted to switch to 0.8 mg/kg monthly (Q4W) at the next cycle following a confirmed ≥PR. Results for these phase 1 prospective cohorts are pooled. Supportive analyses were also performed based on patients in phases 1/2 who received the RP2Ds and switched to reduced dosing based on meeting response criteria or to mitigate treatment-emergent adverse events (TEAEs). Dose reduction could be achieved by reducing dose frequency or by reducing dose. ORRs were assessed per IMWG criteria. TEAEs were graded per CTCAE v4.03.

### Results

In total, 45 patients switched to reduced intensity dosing. As of Jun. 20, 2023, 24 patients were included in the prospective cohorts, with a median follow-up of 9.7 months. In total, 9/12 patients achieved a ≥PR and switched from 0.8 mg/kg Q2W to 0.4 mg/kg Q2W dosing, and 10/12 patients achieved a ≥PR and switched from Q2W to 0.8 mg/kg Q4W dosing. Generally, patients switched to reduced intensity dosing during cycles 3-5. Following the change in dosing, responses deepened in 11/19 patients and were maintained in 5/19 patients; 3/19 patients had disease progression. At 6 months post switch, an estimated 88.9% of responders maintained a response. Oral-related TEAEs, reported in 16/19 (84.2%) patients, improved or resolved in 4 patients 1-6 months after switching to reduced intensity dosing. Nail-related TEAEs, reported in 7/19 (36.8%) patients, improved or resolved in 2 patients after 3-4 months. Skin-related TEAEs, reported in 8/19 (42.1%) patients, resolved in 3 patients after 1-3 months. Overall, improvement or resolution of oral-, nail-, and skin-related TEAEs was observed over time in some patients in the prospective reduced and less frequent dosing cohorts. No patients discontinued tal due to these TEAEs.

As of Jan. 17, 2023, supportive phase 1/2 analyses included 20 patients who switched from tal 0.4 mg/kg QW to a reduced dose (TEAE mitigation, n=16; response, n=3; both, n=1), and 6 patients who switched from tal 0.8 mg/kg Q2W to a reduced dose (TEAE mitigation, n=4; response, n=2). In patients who switched from tal 0.4 mg/kg to a reduced dose, an estimated 84.2% and 78.9% of responders maintained a response at 9 and 12 months, respectively. In patients who switched from tal 0.8 mg/kg Q2W to a reduced dose, an estimated 100% and 80.0% of responders maintained a response at 9 and 12 months, respectively.

### UPDATED Results:

As of 17 Sep. 2023, 17 participants in the pivotal RP2D population for talquetamab in MonumenTAL-1 had decreased their talquetamab dosing frequency from 0.8 mg/kg Q2W to 0.8 mg/kg Q4W due to response and/or management of AEs. For participants who decreased dosing frequency to the Q4W dose, median progression-free survival (PFS) was 17.8 months compared with median PFS of 14.2 months of all participants in MonumenTAL-1 (0.8 mg/kg Q2W). Prior to switching to Q4W dosing, all 17 participants (100%) had experienced 1 or more study drug-related TEAE, whereas study drug-related TEAEs occurred in 12 participants (70.6%) after switching. Grade 3 or 4 events had occurred in 16 participants (94.1%) prior to switching and in 6 participants (35.3%) after switching to Q4W dosing. To further address less frequent talquetamab dosing, a

Phase 1 prospective dose reduction cohort was established in MonumenTAL-1 in which participants received treatment with talquetamab 0.8 mg/kg SC Q2W until response of confirmed PR or better and then decreased the frequency of talquetamab dosing to Q4W. As of 11 Oct. 2023, this cohort has shown a median time to first response of 1.2 months and a median time to best response of 2.2 months for participants treated with talquetamab 0.8 mg/kg SC Q2W. Of the 12 participants enrolled into this cohort, 10 achieved a response of PR or better and had a subsequent change in dosing frequency to 0.8 mg/kg SC Q4W and 2 participants had best response of PD. Dose frequency changes for the 10 participants with response of PR or better occurred on Day 1 of Cycles 3 to 5. Eight of the 10 participants maintained or deepened their response after the change to Q4W dosing. The proposed Q4W dosing schedule of talquetamab would maintain efficacy by balancing maximal reduction of disease burden with biweekly dosing in the initial 6 cycles of treatment with participant convenience and reduced exposure with Q4W dosing from Cycle 7 onward for participants with response of confirmed PR or better (or as early as Cycle 5 for participants who have 5 achieved a confirmed response of VGPR or better).

### Conclusions:

Most patients who switched to reduced intensity dosing in MonumenTAL-1 deepened or maintained responses to Tal. GPRC5D-associated TEAEs generally improved over time in the prospectively design cohorts. Overall, reduced or less frequent tal dosing may help to mitigate these TEAEs while maintaining response.

What is claimed is:

1. A method of treating multiple myeloma in a subject in need thereof, comprising administering to the subject a therapeutically effective amount of a GPRC5DxCD3 bispecific antibody on a monthly (Q4W) dosing schedule.

2. The method of claim 1, wherein the GPRC5DxCD3 bispecific antibody comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

3. The method of claim 1 or 2, wherein the GPRC5DxCD3 bispecific antibody comprises a GPRC5D binding domain comprising a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

4. The method of any of claims 1-3, wherein the GPRC5DxCD3 bispecific antibody is an IgG1, an IgG2, an IgG3 or an IgG4 isotype.

5. The method of any of claims 1-4, wherein the GPRC5DxCD3 bispecific antibody is an IgG4 isotype.

6. The method of any of claims 1-5, wherein the GPRC5DxCD3 bispecific antibody comprises one or more substitutions in its Fc region.

7. The method of any of claims 1-6, wherein the GPRC5DxCD3 bispecific antibody is an IgG4 isotype and comprises S228P, F234A and L235A substitutions in its Fc region (according to EU numbering).

8. The method of any of claims 1-7, wherein the GPRC5DxCD3 bispecific antibody is an IgG4 isotype and comprises S228P, F234A, L235A F405L and R409K substitutions in its Fc region (according to EU numbering).

9. The method of any of claims 1-8, wherein the Fc region of the GPRC5D binding arm comprises S228P, F234A and L235A substitutions in its Fc region (according to EU numbering).

10. The method of any of claims 1-9, wherein the Fc region of the CD3 binding arm comprises S228P, F234A, L235A, F405L, and R409K substitutions in its Fc region (according to EU numbering).

11. The method of any of claims 1-10, wherein the GPRC5DxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

12. The method of any of claims 1-10, wherein the GPRC5DxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

13. The method of any of claims 1-10, wherein the GPRC5DxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

14. The method of any of claims 1-10, wherein the GPRC5DxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

15. The method of any of claims 1-10, wherein the GPRC5DxCD3 bispecific antibody is talquetamab.

16. The method of any of claims 1-15, wherein the subject has relapsed or refractory multiple myeloma myeloma.

17. The method of any of claims 1-16, wherein the subject has received at least three prior lines of therapy.

18. The method of any of claims 1-16, wherein the subject has received at least four prior lines of therapy.

19. The method of any of claims 1-16, wherein the subject has received at least five prior lines of therapy (penta-drug exposed).

20. The method of any of claims 1-16, wherein the subject has received at least three prior lines of therapy, including a proteasome inhibitor, an immunomodulatory agent and an anti-CD38 monoclonal antibody.

21. The method of any of claims 1-16, wherein the subject has received at least four prior lines of therapy, including a proteasome inhibitor, an immunomodulatory agent and an anti-CD38 monoclonal antibody.

22. The method of any of claims 1-21 comprising subcutaneously administering treatment doses of the GPRC5DxCD3 bispecific antibody on the monthly dosing schedule (Q4W).

23. The method of any of claims 1-22 comprising subcutaneously administering to the subject one or more step-up doses of the GPRC5DxCD3 bispecific antibody prior to administering the first treatment dose of the GPRC5DxCD3 bispecific antibody.

24. The method of any of claims 1-23 comprising subcutaneously administering the GPRC5DxCD3 bispecific antibody on the monthly dosing schedule (Q4W) at a treatment dose of from about 400 μg/kg to about 800 μg/kg.

25. The method of any of claims 1-23 comprising subcutaneously administering the GPRC5DxCD3 bispecific antibody on the monthly dosing schedule (Q4W) at a treatment dose of about 400 μg/kg.

26. The method of any of claims 1-23 comprising subcutaneously administering the GPRC5DxCD3 bispecific antibody on the monthly dosing schedule (Q4W) at a treatment dose of about 800 μg/kg.

27. The method of any of claims 1-26 comprising subcutaneously administering to the subject at least one treatment dose of the GPRC5DxCD3 bispecific antibody on a bi-weekly dosing schedule (Q2W) and subsequently administering to the subject treatment doses of the GPRC5DxCD3 bispecific antibody on the monthly dosing schedule (Q4W).

28. The method of any of claims 1-27 comprising subcutaneously administering to the subject at least one treatment dose of the GPRC5DxCD3 bispecific antibody on a weekly dosing schedule (QW) and then subcutaneously administering to the subject at least one treatment dose of the GPRC5DxCD3 bispecific antibody on a bi-weekly dosing schedule (Q2W) and then subsequently administering to the subject treatment doses of the GPRC5DxCD3 bispecific antibody on the monthly dosing schedule (Q4W).

29. The method of any of claims 1-28 comprising subcutaneously administering to the subject treatment doses of the GPRC5DxCD3 bispecific antibody on a bi-weekly dosing schedule (Q2W) for 6 treatment cycles, and starting at treatment cycle 7, administering to the subject treatment doses of the GPRC5DxCD3 bispecific antibody on the monthly dosing schedule (Q4W).

30. The method of any of claims 1-28 comprising subcutaneously administering to the subject treatment doses of the GPRC5DxCD3 bispecific antibody on a bi-weekly dosing schedule (Q2W) for 6 treatment cycles, and if the patient has achieved a VGPR, CR or sCR, as determined by IMWG response criteria, then starting at treatment cycle 7, administering to the subject treatment doses of the GPRC5DxCD3 bispecific antibody on the monthly dosing schedule (Q4W).

31. The method of any of claims 1-28 comprising subcutaneously administering to the subject treatment doses of the GPRC5DxCD3 bispecific antibody on a bi-weekly dosing schedule (Q2W) for 7 treatment cycles, and starting at treatment cycle 8, administering to the subject treatment doses of the GPRC5DxCD3 bispecific antibody on the monthly dosing schedule (Q4W).

32. The method of any of claims 1-28 comprising subcutaneously administering to the subject treatment doses of the GPRC5DxCD3 bispecific antibody on a bi-weekly dosing schedule (Q2W) for 7 treatment cycles, and if the patient has achieved a VGPR, CR or sCR, as determined by IMWG response criteria, then starting at treatment cycle 8, administering to the subject treatment doses of the GPRC5DxCD3 bispecific antibody on the monthly dosing schedule (Q4W).

33. The method of any of claims 1-28 comprising subcutaneously administering to the subject treatment doses of the GPRC5DxCD3 bispecific antibody on a bi-weekly dosing schedule (Q2W) for 8 treatment cycles, and starting at treatment cycle 9, administering to the subject treatment doses of the GPRC5DxCD3 bispecific antibody on the monthly dosing schedule (Q4W).

34. The method of any of claims 1-28 comprising subcutaneously administering to the subject treatment doses of the GPRC5DxCD3 bispecific antibody on a bi-weekly dosing schedule (Q2W) for 8 treatment cycles, and if the patient has achieved a VGPR, CR or sCR, as determined by IMWG response criteria, then starting at treatment cycle 9, administering to the subject treatment doses of the GPRC5DxCD3 bispecific antibody on the monthly dosing schedule (Q4W).

35. The method of any of claims 1-27, comprising subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 7 or treatment cycle 8.

36. The method of any of claims 1-27, comprising subcutaneously administering to the subject treatment doses of the GPRC5DxCD3 bispecific antibody on a bi-weekly dosing schedule (Q2W) and then subcutaneously administering to the subject treatment doses of the GPRC5DxCD3 bispecific antibody on the monthly dosing schedule (Q4W) at treatment cycle 5 if the subject has achieved a complete response or a stringent complete response, as determined by IMWG response criteria.

37. The method of any of claims 27-36 wherein each treatment dose amount of the GPRC5DxCD3 bispecific antibody administered on a weekly dosing schedule (QW), a bi-weekly dosing schedule (Q2W) or the monthly dosing schedule (Q4W), is the same.

38. The method of any of claims 27-36 wherein the GPRC5DxCD3 bispecific antibody is administered on the bi-weekly dosing schedule (Q2W) and the monthly dosing schedule (Q4W), at a treatment dose amount of 800 μg/kg.

39. The method of any of claims 1-38, comprising subcutaneously administering 2 or 3 step-up doses of the GPRC5DxCD3 bispecific antibody prior to subcutaneously administering the first treatment dose.

40. The method of any of claims 1-38, comprising subcutaneously administering step-up doses of 10 μg/kg and 60 μg/kg of the GPRC5DxCD3 bispecific antibody prior to subcutaneously administering a treatment dose.

41. The method of any of claims 1-38, comprising subcutaneously administering step-up doses of 10 μg/kg and 60 μg/kg and 400 μg/kg of the GPRC5DxCD3 bispecific antibody prior to subcutaneously administering a treatment dose.

42. The method of any of claims 1-41, comprising subcutaneously administering step-up doses of the GPRC5DxCD3 bispecific antibody 2-4 days apart from each other.

43. The method of any of claims 1-42, wherein the subject achieves a clinical response that is a PR, or a VGPR, or a CR or a sCR.

44. The method of any of claims 1-42, wherein the subject achieves a clinical response that is a CR or a sCR.

45. The method of any of claims 1-27, comprising subcutaneously administering to the subject treatment doses of the GPRC5DxCD3 bispecific antibody on a bi-weekly dosing schedule (Q2W) and then (i) subcutaneously administering to the subject treatment doses of the GPRC5DxCD3 bispecific antibody on the monthly dosing schedule (Q4W) starting at treatment cycle 5 if the subject has achieved a very good partial response, a complete response or a stringent complete response, as determined by IMWG response criteria; or (ii) subcutaneously administering to the subject treatment doses of the GPRC5DxCD3 bispecific antibody on the monthly dosing schedule (Q4W) starting at treatment cycle 7, regardless of clinical response in the subject.

46. The method of claim 45 wherein the GPRC5DxCD3 bispecific antibody is administered on the bi-weekly dosing schedule (Q2W) and the monthly dosing schedule (Q4W), at a treatment dose amount of 800 μg/kg.

47. The method of any of claims 45-46, comprising subcutaneously administering 2 or 3 step-up doses of the GPRC5DxCD3 bispecific antibody prior to subcutaneously administering the first treatment dose.

48. The method of any of claims 45-47, comprising subcutaneously administering step-up doses of 10 μg/kg and 60 μg/kg of the GPRC5DxCD3 bispecific antibody prior to subcutaneously administering a treatment dose.

49. The method of any of claims 45-48, comprising subcutaneously administering step-up doses of 10 μg/kg and 60 μg/kg and 400 μg/kg of the GPRC5DxCD3 bispecific antibody prior to subcutaneously administering a treatment dose.

50. The method of any of claims 45-49, comprising subcutaneously administering step-up doses of the GPRC5DxCD3 bispecific antibody 2-4 days apart from each other.

51. The method of any of claims 45-50, wherein the subject achieves a clinical response that is a PR, or a VGPR, or a CR or a sCR.

52. The method of any of claims 45-51, wherein the subject achieves a clinical response that is a CR or a sCR.

53. The method of any of claims 1-52, comprising treating the subject according to a therapeutically effective regimen that comprises sequential 28-day GPRC5DxCD3 treatment cycles, wherein:
one or more step-up doses of the GPRC5DxCD3 bispecific antibody are subcutaneously administered to the subject during a step-up phase, and
treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W).

54. The method of any of claims 1-53, comprising treating the subject according to a therapeutically effective regimen that comprises sequential 28-day GPRC5DxCD3 treatment cycles, wherein:
one or more step-up doses of the GPRC5DxCD3 bispecific antibody are subcutaneously administered to the subject during a step-up phase, and
treatment doses of the GPRC5DxCD3 bispecific antibody are subcutaneously administered to the subject on a bi-weekly dosing schedule (Q2W) starting from the first GPRC5DxCD3 treatment cycle after the step-up phase, and then treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

55. The method of claim 53 or 54, wherein each treatment dose of the GPRC5DxCD3 bispecific antibody administered on the bi-weekly dosing schedule (Q2W) and the monthly dosing schedule (Q4W) is 800 μg/kg.

56. The method of any of claims 53-55, wherein Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W).

57. The method of any of claims 53-56, wherein Cycle 1 of the regimen comprises the step-up phase, and the treatment doses of the GPRC5DxCD3 bispecific antibody are subcutaneously administered to the subject on the bi-weekly dosing schedule (Q2W) starting from Cycle 2, and then the treatment doses of the GPRC5DxCD3 bispecific antibody are subcutaneously administered to the subject on the monthly dosing schedule (Q4W) either: (i) starting at treatment Cycle 5 if the subject has achieved a very good partial response, a complete response or a stringent complete response, as determined by IMWG response criteria; or (ii) starting at treatment Cycle 7, regardless of clinical response in the subject.

58. The method of any of claims 53-57, wherein 1-3 step-up doses of the GPRC5DxCD3 bispecific antibody are administered to the subject during the step-up phase.

59. The method of any of claims 53-58, wherein 2 step-up doses of the GPRC5DxCD3 bispecific antibody are administered to the subject during the step-up phase.

60. The method of any of claims 53-58, wherein 3 step-up doses of the GPRC5DxCD3 bispecific antibody are administered to the subject during the step-up phase.

61. The method of any of claims 53-60, wherein the step-up phase comprises a first step-up dose of 0.01 mg/kg, a second step-up dose of 0.06 mg/kg, and a third step-up dose of 0.4 mg/kg.

62. The method of any of claims 53-61, wherein one or more treatment doses of the GPRC5DxCD3 bispecific antibody are subcutaneously administered to the subject during the step-up phase, in addition to the one or more step-up doses (e.g., on a weekly dosing schedule).

63. The method of any of claims 53-61, wherein one or two treatment doses of the GPRC5DxCD3 bispecific antibody are subcutaneously administered to the subject during the step-up phase, in addition to the one or more step-up doses (e.g., on a weekly dosing schedule).

64. The method of any of claims 53-63, wherein Cycle 1 of the regimen comprises a first step-up dose of 0.01 mg/kg, a second step-up dose of 0.06 mg/kg, a third step-up dose of 0.4 mg/kg, and a treatment dose of 0.8 mg/kg.

65. The method of any of claims 53-64, wherein Cycle 1 of the regimen comprises a first step-up dose of 0.01 mg/kg, a second step-up dose of 0.06 mg/kg 2-4 days after the first step-up dose, a third step-up dose of 0.4 mg/kg 4-7 days (or 5-7 days) after the second step-up dose, and a treatment dose of 0.8 mg/kg 5-9 days (or 7-9 days) after the third step-up dose.

66. The method of any of claims 53-65, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15.

67. The method of any of claims 53-66, wherein each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1.

68. The method of any of claims 53-66, wherein either: (i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or (ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

69. The method of any of claims 53-68, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and each of Cycles 2-6 of the regimen comprises a Q2W treatment dose of 0.8 mg/kg on Day 1 and Day 15; and each of Cycles 7+ comprises a Q4W treatment dose of 0.8 mg/kg on Day 1.

70. The method of any of claims 53-68, wherein Cycle 1 of the regimen comprises a first step-up dose of 0.01 mg/kg on Day 1, a second step-up dose of 0.06 mg/kg on Day 3, a third step-up dose of 0.4 mg/kg on Day 8, and a treatment dose of 0.8 mg/kg on Day 15; and either:
(i) each of Cycles 2-6 of the regimen comprises a Q2W treatment dose of 0.8 mg/kg on Day 1 and Day 15, and each of Cycles 7+ comprises a Q4W treatment dose of 0.8 mg/kg on Day 1, or
(ii) each of Cycles 2-4 of the regimen comprises a Q2W treatment dose of 0.8 mg/kg on Day 1 and Day 15, and if the subject has achieved a very good partial response, a complete response or a stringent complete response, as determined by IMWG response criteria, then each of Cycles 5+ comprises a Q4W treatment dose of 0.8 mg/kg on Day 1.

71. The method of any of claims 1-70, wherein a dosing regimen comprising the monthly (Q4W) dosing schedule achieves a lower rate and/or lesser duration of adverse events than a dosing regimen comprising a weekly (QW) and/or bi-weekly (Q2W) dosing schedule and not a monthly (Q4W) dosing schedule.

72. The method of claim 71, wherein the adverse events comprise one or more of oral toxicity (e.g., ageusia, dysgeusia, dry mouth, and/or dysphagia), and/or nail toxicity (e.g., nail disorder), and/or skin toxicity (e.g., dry skin, skin exfoliation, and/or pruritus)

73. The method of any of claims 1-72, wherein a dosing regimen comprising the monthly (Q4W) dosing schedule achieves a stronger and/or longer duration of clinical response than a dosing regimen comprising a weekly (QW) and/or bi-weekly (Q2W) dosing schedule and not a monthly (Q4W) dosing schedule.
